(19) United States
(12) Patent Application Publication
(10) Pub. No.: US 2019/0382476 A1
(43) Pub. Date: Dec. 19, 2019

(54) USE OF TETRASPANIN-2 FOR TREATING DIABETES AND SCREENING AN ANTI-DIABETIC AGENT

(71) Applicant: Korea Basic Science Institute, Daejeon (KR)

(72) Inventors: Ik Soon Jang, Seoul (KR); Kyung Bok Lee, Daejeon (KR); Hyun Jin Jang, Incheon (KR)

(21) Appl. No.: 16/425,551

(22) Filed: May 29, 2019

Related U.S. Application Data
(63) Continuation-in-part of application No. 15/609,605, filed on May 31, 2017.

(30) Foreign Application Priority Data
Sep. 28, 2016 (KR) 10-2016-0124527

(51) Int. Cl. C07K 16/28; A61P 3/10

(52) U.S. Cl. CPC C07K 16/28; A61K 2039/505; A61P 3/10

(57) ABSTRACT

There is provided a method for treating diabetes in a subject in need thereof, the method comprising administering to the subject a composition comprising a tetraspanin-2 (TSPAN2) protein activity inhibitor as an active ingredient in an amount effective for treating diabetes

### CROSS-REFERENCE TO RELATED APPLICATIONS

The present application is a continuation-in-part of U.S. patent application Ser. No. 15/609,605 filed May 31, 2017, which claims the benefit of priority from Korean Patent Application No. 10-2016-0124527 filed on Sep. 28, 2016. The disclosures of the said applications are incorporated by reference as if fully set forth herein.

### BACKGROUND

### Technical Field

Exemplary embodiments relate to a composition using tetraspanin-2 (TSPAN2) for treating diabetes and to a method for screening a diabetes therapeutic agent. In particular, exemplary embodiments relate to a pharmaceutical composition for treating diabetes, the composition containing a tetraspanin-2 (TSPAN2) protein expression or activity inhibitor as an active ingredient. Further, exemplary embodiments relate to a method for treating diabetes, the method comprising administering to a subject in need thereof an effective amount of a tetraspanin-2 (TSPAN2) protein expression or activity inhibitor. Still further, exemplary embodiments relate to a method for screening a diabetes therapeutic agent, the method comprising: (a) contacting a test material with a cell expressing TSPAN2 gene; (b) determining the expression level of the TSPAN2 gene in the cell of (a) and in a control cell which expresses TSPAN2 gene and is not in contact with the test material; and (c) selecting, as a diabetes therapeutic agent candidate, the test material that reduces the expression level of the TSPAN2 gene in comparison with the control cell.

### Discussion of the Background

In diabetes, glucose toxicity leads to β-cell apoptosis and affects various organ systems including pancreas. However, its basic mechanisms have yet to be fully understood. The dysfunction of β-cells and impaired insulin production are the typical features of diabetes. However, despite the growing diabetes epidemic, the exact molecular mechanisms by which glucose toxicity causes β-cell apoptosis are still not fully known.

Tetraspanins are membrane proteins composed of four transmembrane domains and are found in nearly all animal cells. Tetraspanins are involved in fundamental cellular processes, including cell growth, adhesion, and differentiation. Members of the tetraspanin family tend to have highly conserved amino acid sequences. The function and expression of tetraspanin-2 (TSPAN2), however, have not yet been studied.

Apoptosis is a naturally occurring process in the body wherein specialized intracellular signaling is activated, killing the cells. It is a homeostatic mechanism to maintain cell populations in tissues. Fundamentally, improper apoptosis is commonly shown in various pathological phenomena, including glucose toxicity, neurodegenerative diseases, metabolic stress such as ischemic damage, autoimmune disorders, and many types of cancer. The binding of c-Jun and β-catenin/TCF4 to the c-Jun promoter depends upon JNK activity. Therefore, one role of this complex is to mediate cell maintenance, proliferation, differentiation, and apoptosis, whereas the down-regulation of these signals may contribute to carcinogenesis. JNK functions in the noncanonical Wnt pathway to regulate convergent extension movements in vertebrate gastrulation. JNK also prevents nuclear β-catenin accumulation and regulates axis formation in Xenopus embryos. β-catenin enhances the survival of renal epithelial cells by inhibiting BCL2-associated X protein (Bax), which has a proapoptotic function within the cell.

Although the effects of glucose toxicity on β-cell dysfunction and apoptosis have been studied in detail, the precise molecular mechanisms of glucose toxicity have not been elucidated. Glucotoxicity-induced pancreatic β-cell apoptosis by the regulation of the JNK/β-catenin pathway is not yet understood. Therefore, the present inventors investigated the role of the TSPAN2-associated JNK/β-catenin pathway in pancreatic β-cell apoptosis, thereby increasing the understanding of the pathology of diabetes, and there is a need to find diabetes therapeutic agent candidates with new mechanisms.

The above information disclosed in this Background section is only for enhancement of understanding of the background of the inventive concept, and, therefore, it may contain information that does not form the prior art that is already known in this country to a person of ordinary skill in the art.

### DETAILED DESCRIPTION OF THE INVENTION

### Technical Problem

The present inventors have verified that, in pancreatic β-cells under hyperglycemic conditions, TSPAN2 promotes apoptosis through caspase-3 by increasing JNK phosphorylation and inhibiting the nuclear translocation of β-catenin.

Therefore, an aspect of the present invention is to provide a pharmaceutical composition for treating diabetes, the composition comprising a tetraspanin-2 (TSPAN2) protein expression or activity inhibitor as an active ingredient.

Another aspect of the present invention is to provide a method for screening a diabetes therapeutic agent, the method comprising:

(a) contacting a test material with a cell expressing TSPAN2 gene;

(b) determining the expression level of the TSPAN2 gene in the cell of (a) and in a control cell which expresses TSPAN2 gene and is not in contact with the test material; and

(c) selecting, as a diabetes therapeutic agent candidate, the test material that reduces the expression level of the TSPAN2 gene in comparison with the control cell.

### SUMMARY

This Summary lists several embodiments of the presently disclosed subject matter. This Summary is merely exemplary of the numerous and varied embodiments. Mention of one or more representative features of a given embodiments is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features. Additional embodiments will be apparent from the disclosure, or may be learned by practice of the inventive concept.

In one embodiment, the presently disclosed subject matter provides a pharmaceutical composition for treating diabetes, the composition comprising a tetraspanin-2 (TSPAN2) protein expression or activity inhibitor as an active ingredient.

In another embodiment, the presently disclosed subject matter provides a method for treating diabetes, the method comprising administering to a subject in need thereof an effective amount of a tetraspanin-2 (TSPAN2) protein expression or activity inhibitor.

In further embodiment, the diabetes is selected from the group consisting of Type 1 diabetes mellitus, Type 2 diabetes mellitus, and gestational diabetes mellitus.

In some embodiment, the TSPAN2 protein includes the amino acid sequence represented by any one sequence selected from the group consisting of SEQ ID NO: 1 to SEQ ID NO: 3.

In another embodiment, the TSPAN2 protein expression inhibitor is any one selected from the group consisting of an antisense oligonucleotide, siRNA, shRNA, miRNA, ribozyme, DNAzyme, and protein nucleic acid (PNA), each of which complementarily binds to TSPAN2 mRNA.

In still another embodiment, the TSPAN2 mRNA includes the nucleotide sequence represented by any one sequence selected from the group consisting of SEQ ID NO: 4 to SEQ ID NO: 6.

In still another embodiment, the siRNA complementarily binding to the TSPAN2 mRNA includes the nucleotide sequence represented by any one sequence selected from the group consisting of SEQ ID NO: 7 to SEQ ID NO: 33.

In another embodiment, the TSPAN2 protein activity inhibitor is any one selected from the group consisting of a compound, a peptide, a peptide mimetic, an aptamer, an antibody, a natural extract, and a synthetic compound, each of which specifically binds to the TSPAN2 protein.

In still another embodiment, the presently disclosed subject matter provides a method for screening a diabetes therapeutic agent, the method comprising:

(a) contacting a test material with a cell expressing TSPAN2 gene;

(b) determining the expression level of the TSPAN2 gene in the cell of (a) and in a control cell which expresses TSPAN2 gene and is not in contact with the test material; and

(c) selecting, as a diabetes therapeutic agent candidate, the test material that reduces the expression level of the TSPAN2 gene in comparison with the control cell.

In further embodiment, the cell expressing the TSPAN2 gene is a pancreatic β-cell or a cell derived therefrom.

In still further embodiment, the determining the expression level of the TSPAN2 gene comprises determining the expression level of TSPAN2 mRNA or protein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A and FIG. 1B show gene ontology analysis results using a microarray in order to examine genes which respond to glucose stimulation. Lists of genes with two-fold up-regulation (FIG. 1A) and down-regulation (FIG. 1B) in RNAKT-15 cells incubated for 48 h under NG or HG conditions were developed using DAVID for gene ontology analysis.

FIG. 2 shows apoptosis-related genes and analysis results of their hierarchical clustering in response to glucose stimulation. Red and green represent genes of which the expression levels were changed by two-fold or larger.

FIG. 3A, FIG. 3B and FIG. 3C show microarray results confirming the expression level of TSPAN2 (FIG. 3A), western blot results confirming the expression level of TSPAN2 protein (FIG. 3B), and RT-PCR results confirming the expression level of TSPAN2 mRNA (FIG. 3C) in RNAKT-15 cells incubated under NG or HG conditions, respectively.

FIG. 4A, FIG. 4B and FIG. 4C show Annexin V staining results (FIG. 4A), changes in protein levels of caspase-3, cleaved caspase-3, and Bax (FIG. 4B), and western blot results (FIG. 4C) in RNAKT-15 cells incubated under NG or HG conditions, respectively. In FIG. 4B, mark represents a statistically significant difference.

FIG. 5A and FIG. 5B show western blot results confirming the protein levels of p-JNK, JNK, caspase 3, cleaved caspase 3, Bcl-2, t-Bid, Bax, PARP, cleaved PARP, and β-catenin (FIG. 5A) and comparison of protein expression levels (FIG. 5B) in RNAKT-15 cells overexpressing TSPAN2 or under HG condition, respectively. In FIG. 5B, mark “***” represents a statistically significant difference.

FIG. 6A and FIG. 6B show western blot results confirming the protein levels of E-cadherin, TSPAN2, β-catenin, n-β-catenin, Bax (FIG. 6A) and comparison of protein expression levels (FIG. 6B) in RNAKT-15 cells inhibiting the expression of TSPAN2 cells using siRNA or under HG conditions, respectively. In FIG. 6B, mark “***” represents a statistically significant difference.

FIG. 7 shows fluorescence microscopic images confirming the effect of Dkk1 on the nuclear translocation of β-catenin in RNAKT-15 cells under HG conditions. si-Dkk1 indicates siRNA for inhibiting Dkk1 expression. Blue indicates DAPI, while green indicates β-catenin protein.

FIG. 8A and FIG. 8B show western blot results confirming the protein expression levels of Dkk1, p-JNK, and JNK in RNAKT-15 cells under HG or SP600125 conditions (FIG. 8A) and the protein expression level comparison (FIG. 8B), respectively. In FIG. 8B, mark “***” represents a statistically significant difference.

FIG. 9A and FIG. 9B show, through a test for examining the effect of TSPAN2 on the nuclear translocation of β-catenin, western blot results confirming the protein expression levels of nuclear β-catenin (N-β-catenin), total β-catenin, p-Akt, and Akt in HG, TSPAN2 overexpressed, or HG and si-Dkk1-treated RNAKT-15 cells (FIG. 9A) and the protein expression level comparison (FIG. 9B), respectively. In FIG. 9B, mark “***” represents a statistically significant difference.

FIG. 10A and FIG. 10B show, through a test for examining the effect of TSPAN2 on JNK/β-catenin, western blot results confirming the protein expression levels of E-cadherin, p-β-catenin, n-β-catenin, TSPAN2, p-JNK, and JNK, Bax in RNAKT-15 cells stimulated with various combinations of HG, si-TSPAN2, SP600125, and si-β-catenin treatment conditions (FIG. 10A) and the protein expression level comparison (FIG. 10B). In FIG. 10B, mark “***” represents a statistically significant difference.

FIG. 11 shows flow cytometry results of PI staining for confirming apoptosis in RNAKT-15 cells wherein the expression of TSPAN2 was inhibited by si-TSPAN2 for 1 day (siTS2 1d) or 2 days (siTS2 2d) under NG or HG conditions.

FIG. 12 shows the blood glucose level reduction and maintenance effect by administration of anti-TSPAN2 Ab in the type 1 diabetic mouse model.

FIG. 13A and FIG. 13B show the recovery effect of the pancreas and spleen by administration of anti-TSPAN2 Ab in the type 1 diabetic mouse model. FIG. 13A shows the image of the organ, and FIG. 13B shows the results of quantifying the volume of each organ.

FIG. 14 shows blood glucose level reduction and maintenance effects by administration of anti-TSPAN2 Ab in a mouse model of type 2 diabetes.

FIG. 15 shows improvement of glucose tolerance by administration of anti-TSPAN2 Ab in a mouse model of type 2 diabetes.

FIG. 16 shows improvement of insulin sensitivity by administration of anti-TSPAN2 Ab in a mouse model of type 2 diabetes.

### DETAILED DESCRIPTION OF THE ILLUSTRATED EMBODIMENTS

The presently disclosed subject matter will be described more fully hereinafter with reference to the accompanying Examples and Drawings, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments of those skilled in the art.

It will be apparent to one skilled in the art that various modifications and variations can be made in the present invention without departing from the spirit or scope of the invention. Thus, it is intended that the present invention cover the modifications and variations of this invention provided come within the scope of the appended claims and their equivalents.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

In some embodiment, the presently disclosed subject matter provides a pharmaceutical composition for treating diabetes, the composition comprising a tetraspanin-2 (TSPAN2) protein expression or activity inhibitor as an active ingredient.

The term “treating” refers to inhibiting occurrence or recurrence of a disease, alleviating symptoms, reducing direct or indirect pathological consequences of a disease, reducing the progression rate of a disease, improving, alleviating or relieving disease conditions, or improving prognosis, as well as an effect of inhibiting the onset of a disease or delaying the progress of a disease. In particular, the term “treating” broadly refers to the improvement of diabetes or a diabetic condition, or the amelioration of symptoms derived from diabetes or a diabetic condition, while including, without limitation, curing, substantially preventing, and improving diabetes or a diabetic condition, and relieving, curing or preventing one or more of the symptoms resulting from diabetes or a diabetic condition.

As used herein, the term “protein” is used interchangeably with the term “polypeptide” or “peptide”, and refers to, for example, a polymer of amino acid residues, as typically found in natural proteins.

As used herein, the term “polynucleotide” or “nucleic acid” refers to a single-stranded or double-stranded deoxyribonucleic acid (DNA) or ribonucleic acid (RNA). Unless otherwise limited, the term includes known analogs of natural nucleotides that hybridize to nucleic acids in a manner similar to naturally-occurring nucleotides. In general, DNA is composed of four bases, i.e., adenine (A), guanine (G), cytosine (C), and thymine (T), while thymine (T) is replaced by uracil (U) in RNA. In the double-stranded nucleic acid, base A forms a hydrogen bond with base T or U, while base C with base G. Such a relationship between these bases is referred to as being “complementary”.

Meanwhile, “mRNA (messenger RNA)” is RNA that acts as a blueprint for polypeptide synthesis (protein translation) by transferring the genetic information of the nucleotide sequence of a particular gene to the ribosome during protein synthesis. Single-stranded mRNA is synthesized through a transcription process using the gene as a template.

In the present invention, “diabetes”, which is an object of treatment, is a metabolic syndrome characterized by deficiency in pancreatic β-cells-producing insulin hormone or abnormality of insulin resistance, and furthermore, hyperglycemia resulting from both of these defects. Such diabetes may be divided into insulin dependent diabetes mellitus (IDDM, Type 1) and noninsulin dependent diabetes mellitus (NIDDM, Type 2) caused by insulin resistance or insulin secretion damage. Both of Type 1 and Type 2 diabetes mellitus cause various complications, such as heart disease, intestinal disease, ocular disease, neurological disease, and stroke, wherein the blood glucose and insulin levels are increased for a long period of time, resulting in chronic neurological diseases and cardiovascular diseases, with acute complications through a short-term hypoglycemia and hyperglycemia responses. Diabetes causes disorders in lipid and protein metabolisms as well as the carbohydrate metabolism, while hyperglycemia is chronically maintained. The conditions of diabetes are various, while those directly caused by hyperglycemia are diabetic peripheral neuropathy, diabetic retinopathy, diabetic nephropathy, diabetic cataract, keratosis, diabetic atherosclerosis, and the like in the retina, kidney, nerves, and cardiovascular system.

The death of pancreatic β-cells by hyperglycemia is one of the important pathologies that cause and aggravate diabetes. The present inventors have established that, with respect to glucose toxicity (or glucotoxicity) affecting various organs including pancreas while the blood glucose is high, tetraspanin-2 regulates the JNK/β-catenin signaling pathway and promotes apoptosis in human pancreatic β-cells. Therefore, it can be understood that the expression or activity of the tetraspanin-2 protein is inhibited to prevent the apoptosis of pancreas, especially β-cells, due to glucose toxicity, thereby inhibiting the occurrence or progression of diabetes. Accordingly, the diabetes in the present invention may be diabetes mellitus caused, aggravated or advanced by glucose toxicity, and more specifically, may be selected from the group consisting of Type 1 diabetes mellitus, Type 2 diabetes mellitus, and gestational diabetes mellitus.

The term “tetraspanin-2” refers to a protein pertaining to transmembrane 4 superfamily or tetraspanin family and a membrane protein having particular four hydrophobic domains, while this protein plays an important role in the development, activation, growth, and mobility of cells. Human tetraspanin-2 is located on chromosome 1p13.2, and is composed of about eight exons. It is also known as NET3, TSN2, TSPAN2, TSPAN-2, or the like, while three major isoforms thereof are known.

The TSPAN2 protein in the present invention may be derived from a mammal, preferably human. Most preferably, the TSPAN2 protein in the present invention includes any one amino acid sequence selected from human TSPAN2 isoform 1 (NP_005716.2) represented by SEQ ID NO: 1, human TSPAN2 isoform 2 (NP_001295244.1) represented by SEQ ID NO: 2, and human TSPAN2 isoform 3 (NP_001295245.1) represented by SEQ ID NO: 3 (The numbers in each parenthesis indicates said isoforms' NCBI Genbank accession numbers, respectively).

The TSPAN2 protein expression inhibitor may be any one selected from the group consisting of an antisense oligonucleotide, siRNA, shRNA, miRNA, ribozyme, DNAzyme, and protein nucleic acid (PAN), which complementarily bind to TSPAN2 mRNA. In addition, the TSPAN2 mRNA includes any one nucleotide sequence selected from human TSPAN2 mRNA transcript variant 1 (NM_005725.5) represented by SEQ ID NO: 4, human TSPAN2 mRNA transcript variant 2 (NM_001308315.1) represented by SEQ ID NO: 5, and human TSPAN2 mRNA transcript variant 3 (NM_001308316.1) represented by SEQ ID NO: 6 (The numbers in each parenthesis indicates said isoforms' NCBI Genbank accession numbers, respectively). The characteristic features of coding regions (exons) of the human TSPAN2 mRNA transcript variants are confirmed in the sequence information obtained by searching the NCBI database through Genbank accession numbers written in the parentheses.

Furthermore, the TSPAN2 protein expression inhibitor according to the present invention may preferably be siRNA that complementarily binds to TSPAN2 mRNA to induce the degradation of mRNA. Specifically, the siRNA for TSPAN2 may include any one nucleotide sequence selected from the group consisting of SEQ ID NO: 7 to SEQ ID NO: 33.

The “siRNA (small interfering RNA or short interfering RNA or silencing RNA)” is a short double-stranded RNA, which is artificially introduced into cells to induce the degradation of mRNA of a specific gene, thereby preventing the translation of its corresponding protein, leading to RNA interference inhibiting the expression of the gene. SiRNA usually consists of 20 to 25 nucleotides complementary to a specific region of target mRNA. An antisense strand complementary to the target mRNA in the double strands of siRNA in cells binds to target mRNA by binding to an RNA-induced silencing complex (RISC) protein complex, and the argonaute protein in the RISC complex cleaves to degrade the target mRNA, or inhibits the binding between the proteins and ribosome important in the protein translation, thereby inhibiting the expression of a particular gene.

The siRNA according to the present invention may be one that has been subjected to various modifications for improving in vivo stability of oligonucleotides, providing resistance to nuclease, and reducing non-specific immune responses. For the modification of the oligonucleotide, one or more modifications selected from a modification in which the OH group at the 2′ carbon position on the sugar structure in at least one nucleotide is substituted with —CH₃ (methyl), —OCH₃ (methoxy), —NH₂, —F, —O-2-methoxyethyl, —O-propyl, —O-2-methylthioethyl, —O-3-aminopropyl, —O-3-dimethyl aminopropyl, —O—N-methyl acetamido or —O-dimethyl amido oxyethyl; a modification in which oxygen in the sugar structure in the nucleotide is substituted with sulfur; or a modification of nucleotide binding into phosphorothioate, boranophosphate, or methyl phosphonate binding may be used in combination. The modification into a form of peptide nucleic acid (PAN), locked nucleic acid (LNA), or unlocked nucleic acid (UNA) is also possible.

In addition, the TSPAN2 protein activity inhibitor may be any one selected from the group consisting of a compound, a peptide, a peptide mimetic, an aptamer, an antibody, a natural extract, and a synthetic compound, which specifically bind to the TSPAN2 protein, but is not limited thereto.

The peptide mimetics may be peptides or non-peptides and may include amino acids linked by non-peptide bonds such as psi bonds. Moreover, the peptide mimetics may be “conformationally constrained” peptides or cyclic mimetics including at least one exocyclic domain, a link moiety (linking amino acid) and an active region. The peptide mimetics are constructed to resemble secondary structural features of Ubiquitin-Associated Protein 2 (UBAP2) and may mimic inhibitory features of macro molecules such as antibody or water soluble receptors. These peptides represent novel small molecule that may act with potency equivalent to the natural antagonist. The peptide mimetics inhibit the binding domain of TSPAN2 protein, thus inhibiting the activity of TSPAN2 protein.

The aptamer is a single-stranded DNA or RNA molecule and may be obtained by isolating oligomers that bind to specific chemical molecules or biological molecules with high affinity and specificity by an evolutionary method using an oligonucleotide library called systematic evolution of ligands by exponential enrichment (SELEX). The aptamer may specifically bind to a target to regulate its activity and may inhibit the function of the target by binding, for example.

The antibody specifically and directly binds to the TSPAN2 to effectively inhibit its activity. The “antibody” means immunoglobulin that specifically binds to an antigenic site. The antibody in the present invention is an antibody that specifically binds only to the TSPAN2 protein without reacting with other proteins (including other types of aminoacyl thiourea synthetase other than TSPAN2).

The antibodies against the TSPAN2 may be easily prepared in accordance with conventional technologies known to one skilled in the art. First, it may include cloning the TSPAN2 gene into an expression vector, thereby obtaining a protein encoded by the gene. A fragment of TSPAN2 protein including a TSPAN2 antigenic site, as well as whole TSPAN2 protein, may be used to prepare a TSPAN2 protein specific antibody. Polyclonal antibodies may be prepared by a method widely known in the art, which includes injecting the TSPAN2 protein (or antigenic fragment thereof) into an animal and collecting blood samples from the animal to obtain serum containing antibodies. Such polyclonal antibodies may be prepared from a certain animal host, such as goats, rabbits, sheep, monkeys, horses, pigs, cows and dogs.

Monoclonal antibodies may be prepared by a method widely known in the art, such as a hybridoma method or a phage antibody library technique.

The form of the antibody of the present invention is not particularly limited and includes a polyclonal antibody or a monoclonal antibody. In addition, if it has an antigen-antibody binding property, some (Especially functional fragments) of the whole antibodies are also included in the antibodies of the present invention, and includes all kinds of immunoglobulin antibodies that specifically bind to the TSPAN2. For example, it includes a full form (complete form) antibody having two full-length light chains and two full-length heavy chains, as well as functional fragments of antibody molecules. The functional fragments of antibody molecules refer to fragments retaining at least an antigen-binding function, and include Fab, F(ab′), F(ab′)2 and Fv.

Further, the antibody of the present invention includes a specific antibody such as a humanized antibody, a chimeric antibody and a recombinant antibody as long as it can specifically bind to the TSPAN2 protein.

In one embodiment of the invention, the TSPAN2 protein expression inhibitors or activity inhibitors may be provided in a form that is loaded into the exosome.

In a specific example, The antibody is provided in form of antibody-loaded exosome. The exogenous substances (ex. protein and/or peptide especially antibody) can be introduced into the exosomes by a number of different techniques. In one embodiments of the invention, the exosomes may be loaded with exogenous protein and/or peptide (antibody) using a transfection reagent. Despite the small size of the exosomes, conventional transfection agents may be used for transfection of exosomes with protein and/or peptide. A commercially available transfection reagent can be used. For example, Exo-Fect™ can be used.

Alternatively, the exosomes are loaded by electroporation or the use of a transfection reagent. Electroporation conditions may vary depending on the charge and size of the biotherapeutic cargo. Typical voltages are in the range of 20V/cm to 1000V/cm, such as 20V/cm to 100V/cm with capacitance typically between 25 μF and 250 μF, such as between 25 μF and 125 μF. A voltage in the range of 150 mV to 250 mV, particularly a voltage of 200 mV is may preferred for loading exosomes with an antibody according to the present invention.

In another embodiment, exosomes may also be loaded by transforming or transfecting a host cell with a nucleic acid construct which expresses therapeutic protein or peptide of interest, such that the therapeutic protein or peptide is taken up into the exosomes as the exosomes are produced from the cell.

Other methods of loading exogenous substances into exosomes are understood based on what is known in the art (for example, see document US 2014/0356382 A1 as reference).

The pharmaceutical composition according to the present invention may be variously formulated depending on the route of administration together with a pharmaceutically acceptable carrier for the treatment of diabetes by a method known in the art. The carrier includes all types of solvents, dispersion media, oil-in-water or water-in-oil emulsions, aqueous compositions, liposomes, microbeads, and microsomes.

The pharmaceutical composition according to the present invention may be administered to a patient in a pharmaceutically effective amount, that is, an amount sufficient to prevent diabetes or alleviate and treat symptoms thereof. For example, a general daily dose may be in the range of about 0.01 to 1000 mg/kg, and preferably in the range of about 1 to 100 mg/kg. The pharmaceutical composition of the present invention may be administered once or divided into multiple doses within a desired dose range. The dose of the pharmaceutical composition according to the present invention can be appropriately selected by a person skilled in the art depending on the route of administration, subject to be administered, age, gender, weight, individual difference, and disease state.

The route of administration may be oral or parenteral. The parenteral administration method includes, but is not limited to, intravenous, intramuscular, intraarterial, intramedullary, intradermal, intracardiac, transdermal, subcutaneous, intraperitoneal, intranasal, intestinal enteric, intrapancreatic, topical, sublingual, or rectal administration.

The pharmaceutical composition of the present invention, when orally administered, may be formulated, together with a suitable carrier for oral administration, in the form of a powder, granules, a tablet, a pill, a sugar coated tablet, a capsule, a liquid, a gel, a syrup, a suspension, a wafer, or the like, by a method known in the art. Examples of the suitable carrier may include: sugars including lactose, dextrose, sucrose, sorbitol, mannitol, xylitol, erythritol, and maltitol; starches including corn starch, wheat starch, rice starch, and potato starch; celluloses including cellulose, methyl cellulose, sodium carboxy methyl cellulose, and hydroxypropyl methyl cellulose; and a filler, such as gelatin or polyvinyl pyrrolidone. In some cases, cross-linked polyvinyl pyrrolidone, agar, alginic acid, or sodium alginate may be added as a disintegrant. Further, the pharmaceutical composition of the present invention may further contain an anti-coagulant, a slipping agent, a wetting agent, a favoring agent, an emulsifier, and a preservative.

As for the parenteral administration, the pharmaceutical composition of the present invention may be formulated in a dosage form of an injection, a transdermal administration preparation, and a nasal inhalant, together with a suitable parenteral carrier, by a method known in the art. The injection needs to be essentially sterilized, and be protected from the contamination of microorganisms, such as bacteria and fungi. Examples of the suitable carrier for the injection may include, but are not limited to, water, ethanol, polyols (e.g., glycerol, propylene glycol, liquid polyethylene glycol, etc.), mixtures thereof, and/or solvents or dispersive media containing vegetable oils. More preferably, Hanks' solution, Ringer's solution, phosphate buffered saline (PBS) or sterile water containing triethanolamine for injection, or an isotonic solution (such as 10% ethanol, 40% propylene glycol, or 5% dextrose) may be used as a suitable carrier. In order to protect the injection from microbial contamination, the injection may further contain various antibiotics and antifungal reagents, such as paraben, chlorobutanol, phenol, sorbic acid, and thimerosal. In most cases, the injection may further contain an isotonic agent, such as sugar or sodium chloride.

The form of the transdermal administration preparation includes ointment, cream, lotion, gel, solution for external application, paste, liniment, and aerosol. The “transdermal administration” means locally administering a pharmaceutical composition to skin to deliver an effective amount of an active ingredient through the skin. For example, the pharmaceutical composition of the present invention may be administered by a method of being made into an injection dosage form and slightly pricking the skin with a 30-gauge needle or being directly applied to the skin. These preparations are described in the document, which is a formulary generally known in pharmaceutical chemistry.

In the case of an inhalation administration agent, the compound used according to the present invention may be conveniently delivered in the form of aerosol spray from a pressurized pack or a nebulizer, using a suitable propellant, for example, dichlorofluoromethane, trichlorofluoromethane, dichlorotetrafluoroethane, carbon dioxide, or other suitable gases. In the case of a pressurized aerosol, the dosage unit may be determined by providing a valve that delivers a measured quantity. For example, a gelatin capsule and a cartridge used in an inhaler or an insufflator may be formulated to contain a compound, and a powder mixture of proper powder materials, such as lactose or starch.

The pharmaceutical composition according to the present invention may further contain at least one buffer (for example, saline solution, or PBS), a carbohydrate (for example, glucose, mannose, sucrose, or dextran), a stabilizer (for example, sodium bisulfate, sodium sulfite, or ascorbic acid), an antioxidant, a bacteriostat, a chelating agent (for example, EDTA or glutathione), an adjuvant (for example, aluminum hydroxide), a suspension agent, a thickener, and/or a preservative).

In addition, the pharmaceutical composition of the present invention may be formulated by a method known in the art to provide rapid, continuous, or delayed release of an active ingredient after the pharmaceutical composition is administered to a mammal.

In addition, the pharmaceutical composition of the present invention may be administered alone or administered in combination with a known compound protecting pancreatic cells and possessing a therapeutic effect on diabetes.

In some embodiment, the presently disclosed subject matter provides a method for treating diabetes, the method comprising administering to a subject in need thereof an effective amount of a tetraspanin-2 (TSPAN2) protein expression or activity inhibitor.

As used herein, the term “effective amount” refers to an amount that, when administered to a subject, leads to the effect of improvement, amelioration, treatment, prevention, detection or diagnosis of diabetes. The effective amount varies depending on the route of administration, the severity of disease, sex, weight, age of the subject and the like. One skilled in the art where the presently described subject matter belongs will be able to determine the appropriate effective amount of a tetraspanin-2 (TSPAN2) protein expression or activity inhibitor to be administered in view of the above mentioned various factors.

The term “subject” refers to an animal, preferably a mammal which especially includes a human, while including animal-derived cells, tissues, organs and the like. The subject may be a patient in need of the above mentioned effect.

As used herein, the term “treating” broadly refers to the improvement of diabetes or a diabetic condition, or the amelioration of symptoms derived from diabetes or a diabetic condition, while including, without limitation, curing, substantially preventing, and improving diabetes or a diabetic condition, and relieving, curing or preventing one or more of the symptoms resulting from diabetes or a diabetic condition.

In some embodiment, the presently disclosed subject matter provides a method for treating diabetes in a subject in need thereof, the method comprising administering to the subject a composition comprising a tetraspanin-2 (TSPAN2) protein activity inhibitor as an active ingredient in an amount effective for treating diabetes.

In another embodiment, the presently disclosed subject matter provides a method for treating diabetes in a subject in need thereof, the method comprising administering to the subject a composition consisting of a tetraspanin-2 (TSPAN2) protein activity inhibitor as an active ingredient in an amount effective for treating diabetes.

In another embodiment, the presently disclosed subject matter provides a method for treating diabetes in a subject in need thereof, the method comprising administering to the subject a composition essentially consisting of a tetraspanin-2 (TSPAN2) protein activity inhibitor as an active ingredient in an amount effective for treating diabetes.

The term “comprising” of the present invention is used synonymously with “including” or “characterized” and does not exclude additional component elements or method steps, etc not mentioned in the composition or method. The term “consisting of” is intended to exclude additional elements, steps or components, etc not otherwise mentioned. The term “essentially consisting of” is intended to encompass component elements or steps, etc., which, in addition to the described component elements or steps, do not materially affect the active ingredient (for instance, TSPAN2 inhibitor in the present invention) underlying properties in the scope of the composition or method.

In some embodiment, the presently disclosed subject matter provides a method for screening a diabetes therapeutic agent, the method comprising:

(a) contacting a test material with a cell expressing TSPAN2 gene;

(b) determining the expression level of the TSPAN2 gene in the cell of (a) and in a control cell which expresses TSPAN2 gene and is not in contact with the test material; and

(c) selecting, as a diabetes therapeutic agent candidate, the test material that reduces the expression level of the TSPAN2 gene in comparison with the control cell.

In step (a), a test material, which is a subject of analysis, is contacted with a cell expressing TSPAN2 gene in order to examine whether the test material possesses the effect of inhibiting the expression of TSPAN2 gene.

As used herein, the term “contacting” has a general meaning, and refers to combining two or more agents (e.g., two polypeptides) or combining an agent and a cell (e.g., protein and cell). The contacting may occur in vitro. For example, two or more agents are combined with each other, or a test agent and cells, or a test agent and a cell lysate are combined with each other in a test tube or other container. In addition, the contacting may occur inside a cell or in situ. For example, recombinant polynucleotides encoding two polypeptides are co-expressed in a cell, so that two polypeptides are brought into contact with each other in a cell or a cell lysate. In addition, a protein chip or a protein array in which a protein to be tested is arranged on a surface of a solid phase.

In addition, the “test material” in the above method of the present invention can be used exchangeably with a test agent or an agent, and includes any substance, molecule, element, compound, entity, or a combination thereof. For example, the test material includes a protein, a polypeptide, a small organic molecule, a polysaccharide, a polynucleotide, and the like. Moreover, the test material may be a natural product, a synthetic compound, a chemical compound, or a combination of two or more materials.

More specifically, the test agent that can be screened by the screening method of the present invention includes polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, saccharides, fatty acids, purine, pyrimidine, or derivatives, structural analogs, or combinations thereof. The test agent may be a synthetic or natural material. The test agent may be obtained from a wide variety of sources including synthetic or natural compound libraries. Combinatorial libraries may be produced from several kinds of compounds that can be synthesized in a step-by-step manner. Compounds of multiple combinatorial libraries may be constructed by the encoded synthetic libraries (ESL) method (WO 95/12608, WO 93/06121, WO 94/08051, WO 95/395503, and WO 95/30642). Peptide libraries may be constructed by a phage display method (WO 91/18980). Libraries of natural compounds in the form of bacteria, mold, plant, and animal extracts may be obtained from commercial sources or collected from fields. The known pharmacological agents may be applied to directed or random chemical modifications, such as acylation, alkylation, esterification, and amidification, in order to prepare their structural analogs.

The test agent may be a naturally occurring protein or a fragment thereof. This test agent may be obtained from a natural source, for example, a cell or tissue lysate. The library of polypeptide agents may also be obtained, for example, from cDNA libraries, which are constructed by routine methods or are commercially available. The test agent may be a peptide, such as a peptide having about 5-30 amino acids, preferably about 5-20 amino acids, and more preferably about 7-15 amino acids. The peptide may be a cleaved product of a naturally occurring protein, a random peptide, or a “biased” random peptide. Alternatively, the test agent may be a nucleic acid. The nucleic acid test agent may be a naturally occurring nucleic acid, random nucleic acid, or “biased” random nucleic acid. For example, the cleaved product of a prokaryotic or eukaryotic genome may be used similar to the disclosure above.

In addition, the test agent may be a small molecule (e.g., a molecule with a molecular weight of about 1,000 Da or less). The high-throughput assay may preferably be applied for screening a small-molecule modulator. A variety of assays are useful for the screening.

As used herein, the term “expression” means the formation of protein or nucleic acid in a cell. The TSPAN2-expressing cell may be a cell that expresses TSPAN2 intrinsically or may be a cell that is transfected with a recombinant expression vector containing a polynucleotide encoding TSPAN2 to overexpress TSPAN2. Preferably, the cell expressing the TSPAN2 gene may be a pancreatic β-cell or a cell derived therefrom. The present inventors have used an RNAKT-15 cell line derived from a human pancreatic β-cell as a cell that expresses TSPAN2 intrinsically.

In step (b), the expression level of TSPAN2 gene is determined between in a cell that expresses TSPAN2 and is in contact with the test material and in a control cell that expresses TSPAN2 and is not in contact with the test material.

The method for determining the expression level of the TSPAN2 gene may be conducted by determining the mRNA or protein level of TSPAN2.

For the determination of mRNA expression, any conventional methods for evaluating the expression level may be employed. Examples of the analysis method may include reverse transcription polymerase chain reaction (RT-PCR), competitive RT-PCR, real-time RT-PCR, RNase protection assay (RPA), northern blotting, DNA microarray chip, RNA sequencing, or the like, but are not limited thereto.

In addition, for the method for determining the expression level of a protein, any methods known in the art may be used without limitation. Examples of the method may include western blotting, dot blotting, enzyme-linked immunosorbent assay, radioimmunoassay (RIA), radial immunodiffusion, Ouchterlony immunodiffusion, rocket immunoelectrophoresis, immunohistochemical staining, immunoprecipitation, complement fixation analysis, flow cytometry (FACS), or a protein chip method, but are not limited thereto.

In step (c), a test material that reduces the expression level of the TSPAN2 gene in comparison with a control cell is selected as a diabetes therapeutic agent candidate.

As established by the present inventors, TSPAN2 plays a role in promoting apoptosis of pancreatic β-cells in a hyperglycemic state. Therefore, the test material that reduces the expression level of TSPAN2 gene lowers the protein level of TSPAN2 and/or the activity level of the TSPAN2 protein, thereby preventing apoptosis caused by glucose toxicity in pancreatic β-cells, thus delaying the development of diabetes or alleviating symptoms thereof.

Therefore, the present invention provides: a pharmaceutical composition for treating diabetes, the composition comprising a tetraspanin-2 (TSPAN2) protein expression or activity inhibitor as an active ingredient; a method for treating diabetes, the method comprising administering to a subject in need thereof an effective amount of a tetraspanin-2 (TSPAN2) protein expression or activity inhibitor; and a method for screening a diabetes therapeutic agent by screening, as a diabetes therapeutic agent candidate, a test material reducing the expression level of TSPAN2 gene. In pancreatic β-cells under high glucose conditions, TSPAN2 promotes apoptosis through caspase-3 by increasing the JNK phosphorylation and inhibiting the nuclear translocation of β-catenin. Conversely, the activity of TSPAN2 is lowered by, for instance, inhibiting the expression of TSPAN2, thereby inhibiting apoptosis due to hyperglycemia and protecting pancreatic β-cells. Based on such a mechanism, anti-diabetic candidates can be screened by screening an agent inhibiting the expression or activity of TSPAN2.

Hereinafter, the present invention will be described in detail. However, the following examples are merely for illustrating the present invention and are not intended to limit the scope of the present invention.

Methods

Cell Culture

Human pancreatic β-cell line RNAKT-15 was obtained from Gachon University. Cells were grown using DMEM containing 5 mM glucose, 10% FBS, 1% penicillin-streptomycin, 10 mM nicotinamide, 10 μM troglitazone, and 16.7 μM zinc sulfate under conditions of 37° C. and 5% CO₂. Glucose toxicity was induced by treatment with 33 mM glucose for 48 hours. Thereafter, the cells were treated with 10 μM JNK inhibitor (SP600125, Sigma) for 48 hours.

Microarray Analysis

Human whole-genome microarrays were used for transcription profiling analysis. For control and test RNAs, the synthesis of target cRNA probes and hybridization were performed using a Low RNA Input Linear Amplification kit (Agilent) according to the manufacturer's instructions. In brief, 1 μg of total RNA from each sample was mixed with a T7 promoter primer mix and incubated at 65° C. for 10 min. The cDNA master mix composed of 5× first stand buffer, 0.1 M DTT, 10 mM dNTP mix, and RNase-Out was then added to the reaction mixture, followed by incubation at 40° C. for 2 h. dsDNA synthesis was terminated by incubation at 65° C. for 15 min. After incubation at 40° C. for 2 h, transcription of dsDNA was performed by the addition of the transcription master mix to dsDNA reaction samples. Amplified and labeled cRNA was purified using a cRNA Cleanup Module (Agilent) according to the manufacturer's instructions. Labeled cRNA was quantified using an ND-1000 spectrophotometer (Nano Drop Technologies). cRNA was fragmented by adding 10× blocking agent and 25× fragmentation buffer to cRNA, followed by incubation at 60° C. for 30 min. The fragmented cRNA was resuspended with 2× hybridization buffer, and pipetted directly onto microarrays (44K). The arrays were then hybridized at 65° C. for 17 h using an Agilent Hybridization Oven (Agilent). The hybridized microarrays were washed according to the manufacturer's washing protocol, and fluorescent images were quantified and normalized as described above. The microarray data have been submitted to the Gene Expression Omnibus database (GEO assession number GSE76189).

Data Analysis

Hybridized images were scanned using an Agilent DNA microarray scanner and quantified using Feature Extraction software (Agilent). Data normalization and determination of fold changes in gene expression levels were performed using Gene-SpringGX 7.3 (Agilent). Briefly, the averages of normalized ratios were calculated by dividing the average of the normalized signal channel intensity by the average of the normalized control channel intensity. Functional annotation of genes was performed using Gene Ontology Consortium data (www.geneontology.org/index.shtml) with GeneSpringGX 7.3. Gene classification was based on queries registered in BioCarta (www.biocarta.com), GenMAPP (www.genmapp.org), DAVID (david.abcc.ncifcrf.gov), and Medline databases (www.ncbi.nlm.nih.gov).

Gene Ontology-Related Network Analysis

Bioinformatics gene network analyses were performed using Ingenuity Pathway Analysis (IPA; www.ingenuity.com) to examine the biologic functions of the differentially regulated genes and proteins according to ontology-related interaction networks, including apoptosis signaling. Ingenuity pathway analysis provides protein interaction networks based on the regularly updated “Ingenuity Pathways KnowledgeBase”. This network is optimized to include as many proteins from the input expression profile as possible and aims to produce highly connected networks

Flow Cytometric Analysis Using Annexin v Staining

To detect apoptosis, annexin V and propidium iodide (PI) were analyzed by staining with an Annexin V-FLUOS staining kit (Roche). Cells treated with normal glucose (NG, 15 mM) and high glucose (HG, 33 mM) for 48 h were washed twice with PBS, and then obtained. The obtained cell suspension was centrifuged at 2000 g for 2 min and incubated with 0.2 mg/ml annexin V fluorescence or 1.4 mg/ml PI for 15 min at room temperature. Analyses were performed using a MoFlo Astrios flow cytometer (Beckman Coulter) at a 488 nm stimulation with a 530/30 nm bandpass filter for annexin V detection. Data were analyzed by Summit 6.0 software.

Acquired Nuclear Fraction

RNAKT-15 cells were prepared by incubation with a HG medium for 2 d. The cells obtained were added to 2 ml of homogenization buffer A (25 mM Tris, pH 7.5, 2 mM EDTA, 0.5 mM EGTA, 1 mM DTT, protease inhibitor, 1 mM PMSF, and 0.02% TritonX-100), homogenized 15 times using a 15 ml Dounce homogenizer with a pestle, and centrifuged at 100,000 g for 30 min. The supernatant cytosolic fraction was transferred into a new tube, and 500 [symbol] ml of homogenization buffer B (homogenization buffer A containing 1% Triton X-100) was added to the pellet. The pellet was resuspended by sonication, and incubated for 30 min at 4° C. The protein contents of the cytosolic and nuclear fractions were quantified by using a bicinchoninic acid (BCA) assay kit (Thermo) and analyzed by Western blot analysis.

Western Blot Analysis

Cells were lysed using RIPA buffer (50 mM Tris-HCl, 150 mM NaCl, 1% Nonidet P-40, 0.5% sodium deoxycholate, 0.1% SDS) containing a protease inhibitor cocktail (Roche Diagnostics). The protein constituents of cell lysates were quantified by a BCA protein assay kit (Roche Diagnostics), while cell lysates containing equal amounts of protein were then separated by SDS-PAGE, and then transferred onto Immobilon NC membranes. Blocking was then conducted for 1 h using TBS containing 5% skim milk or 5% bovine serum albumin (BSA) and Tween 20 solution (0.05% Tween 20). Antibodies against TSPAN2 (1:1000; Abnova,), GAPDH (1:5000; Santa Cruz Biotechnology), E-cadherin (1:1000; Cell Signaling Technology), caspase-3 (1:800; Cell Signaling), b-catenin (1:1000; Santa Cruz), phosphorylated(p) b-catenin (Ser33/37/Thr41, 1:200; Cell Signaling), JNK (1:1000; Santa Cruz), phosphorylated JNK (p-JNK; 1:500; Santa Cruz), truncated Bid (t-Bid; 1:1000; Santa Cruz), poly(adenosine diphosphate-ribose) polymerase (PARP; 1:2000; SantaCruz), Bax (1:1000; Abcam), Akt (1:1000; Cell Signaling), phosphorylated Akt (p-Akt; 1:500; Cell Signaling), B-cell CLL/lymphoma 2 (Bcl-2; 1:6000; Cell Signaling), and Dickkopf-1 (Dkk1; 1:500; Cell Signaling) were incubated with probes at 4° C. overnight, and incubated with peroxidase-conjugated secondary antibodies for 1 h. The membranes were rinsed 3 times with TBS and Tween 20 for 5 min each, and bands were observed by ChemiDoc MP system (Bio-Rad) using chemiluminescence system (Thermo Fisher Scientific). The intensity of the bands was measured using Image J software.

Plasmid, RNA Interference, and Quantitative RT-PCR

The TSPAN2 plasmid construction kit was purchased from GenScript. pcDNA 3.1 (Invotrogen) was used as a general expression vectors, while siRNAs were purchased from ST Pharm. The nucleotide sequence for TSPAN2 siRNA is described as sequence listings of SEQ ID NO: 7 to SEQ ID NO: 33. The transfection of siRNA into RNAKT-15 cells was performed using Lipofectamine RNAiMax reagent (Thermo Fisher Scientific) in accordance with the manufacturer's instructions. RT-PCR was performed to compare the relative amounts of mRNAs in cells cultured in NG and HG media. RT was performed in a total volume of 20 ul using Superscript III reverse transcriptase (Thermo Fisher Scientific) according to the manufacturer's protocol. Subsequently, 4 ul of the final RT product mix was PCR amplified. GAPDH was used as a standard, and the sequences of used primer pairs are described in sequence listings in the present specification.

Immunofluorescence Microscopy

RNAKT-15 cells were seeded at 4×10⁴ cells/well on a coverslip in a 12-well plate. After 24 h, the control cells were treated with high-concentration glucose (HG, 33 mM) for 48 h. TSPAN2 siRNA (25 ug)-treated cells were incubated for 24 h and then treated with high-concentration glucose (HG, 33 mM) for 48 h. Cells were washed twice with PBS, and fixed with 4% formaldehyde for 15 min. Cells were treated with 0.1% Triton X-100 for 15 min, and were then blocked with 3% BSA for 1 h. The cells were incubated with primary antibody β-catenin monoclonal purified mouse IgG1 (Abnova) diluted to 1:100 in 3% BSA overnight at 4° C. After 5 times of washes with PBS for 5 min each, the cells were incubated in the dark for 1 h with FITC-anti-mouse (Thermo Fisher Scientific) diluted to 1:200 in 3% BSA. Images of cover slipped cells were acquired by confocal microscope (LSM 710; Carl Zeiss GmbH) equipped with a C-Apochromat 403/1.2 water immersion lens (488 nm argon laser/505-550 nm detection range). Data were analyzed using Zen 2009 Light Edition software (Carl Zeiss).

Statistical Analysis

All results are expressed as means±standard deviation (means±SD) of at least 3 independent experiments. Statistical significance was determined using Student's t test using Prism 5.0 software (GraphPad Software). Values of p≤0.05 were considered statistically significant.

### Example 1: Differential Expression of Genes According to Glucose Concentration

To identify the genes of which expression changed in response to the glucose concentration, microarray analysis was conducted.

RNAKT-15 cells were incubated for 2 d in conditions of normal glucose (NG) or high glucose (HG), respectively, and the changes in gene expression were investigated through microarray method.

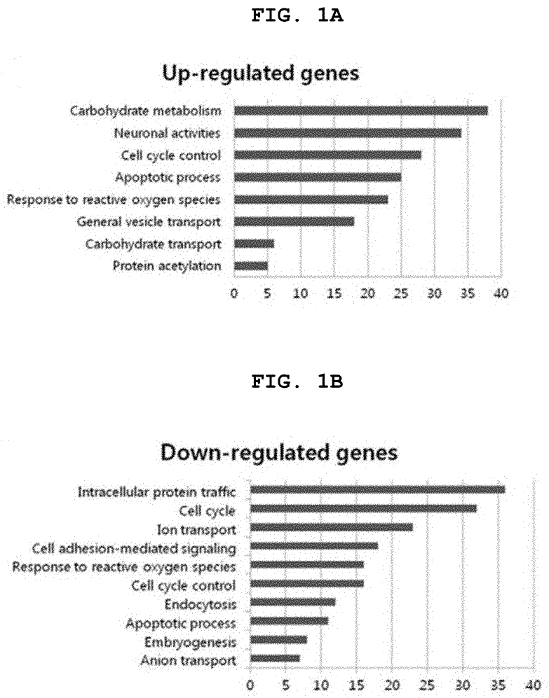

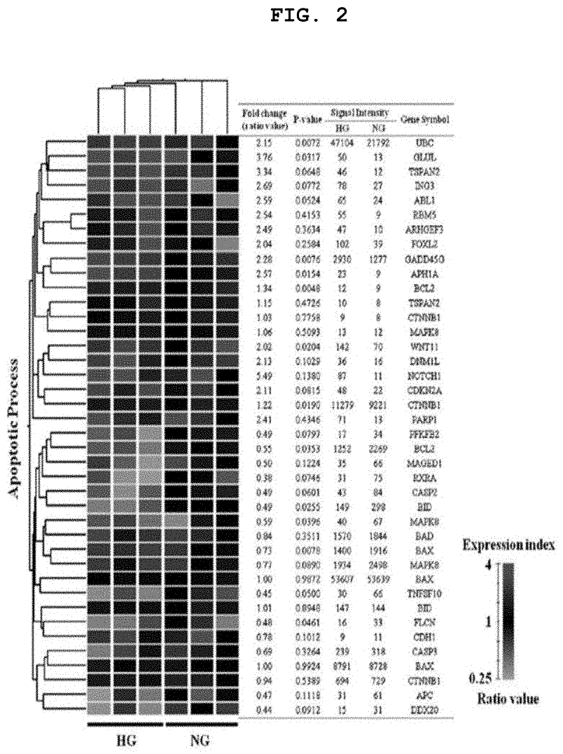

As a result, as shown in FIG. 1A and FIG. 1B, in HG-treated RNAKT-15 cells, the genes of which expression levels were up-regulated and down-regulated were observed, respectively. The genes that were up-regulated were in the following order: carbohydrate metabolism>cell cycle>control>apoptotic processes>response to reactive oxygen species. The genes that were down-regulated were in the following order: intracellular protein trafficking>cell cycle>cell adhesion-mediated signaling>cell cycle control>response to reactive oxygen species>apoptotic process.

To analyze the genes potentially involved in apoptosis, the association between apoptosis and genes that had a 2-fold change in HG compared with NG was separately analyzed (FIG. 2). The intersection obtained by hierarchical clustering is presented with the lists in FIG. 2. Furthermore, as a result of analysis of the signal network system of the genes in response to glucose, it was observed that the expression of apoptosis-related genes was also regulated by glucose (data not shown). Microarray analysis showed that the expression of TSPAN2 increased at least 3-fold under the HG condition, and the signal network analysis suggested that TSPAN2 is involved in cellular apoptosis.

### Example 2: Effect of High Glucose on TSPAN2 Expression and Apoptosis

The effect of TSPAN2 on glucose toxicity-induced β-cell apoptosis was examined.

2-1. Effect of High Glucose on TSPAN2 Expression

The effect of high glucose on TSPAN2 expression was examined.

RNAKT-15 cells were incubated under normal glucose (NG) or high glucose (HG) conditions for 2 d. RNA was extracted from the cells, and transcriptional profiling was performed using microarrays. Microarray experiments were repeated three times, and data analysis was performed. In addition, for analysis of gene expression, RNAKT-15 cells were incubated in normal glucose and high glucose media, respectively, for 2 d, and then cell lysates were subjected to Western blot analysis using anti-TSPAN2 antibody. RT-PCR was conducted to determine the expression levels of TSPAN2 mRNA.

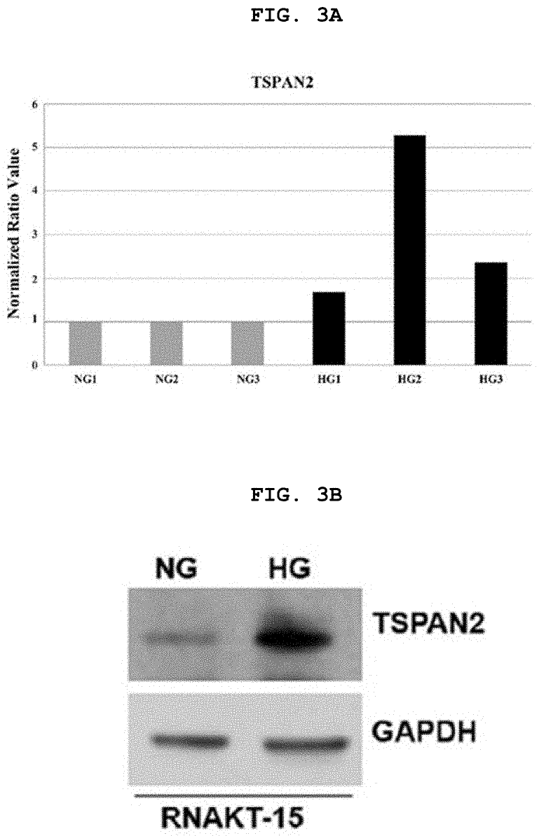

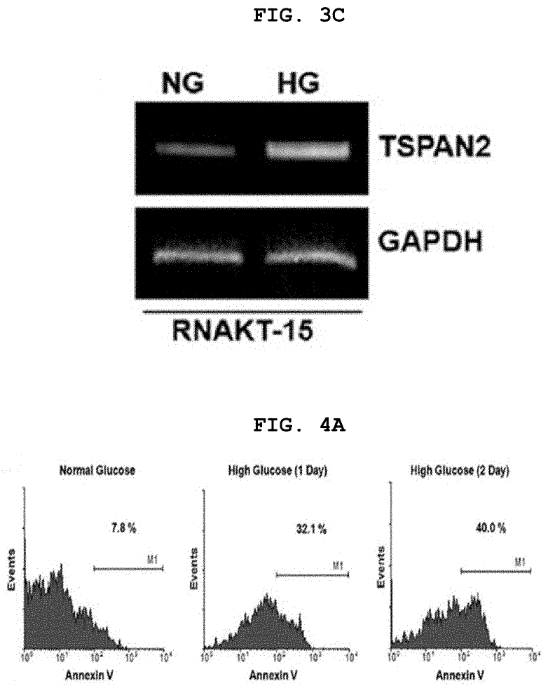

As a result, FIG. 3A to 3C revealed that the expression of TSPAN2 was consistently increased in high glucose conditions. In high glucose conditions, the expression of TSPAN2 was increased up to 3-fold and the expression of its mRNA was increased up to 4-fold.

2-2. Effect of TSPAN2 on Apoptosis

The relationship between TSPAN2 and apoptosis was examined.

RNAKT-15 cells were incubated in normal glucose (NG) or high glucose (HG) conditions, respectively, for 2 d. Thereafter, the cells were stained with annexin V, and observed by a microscope.

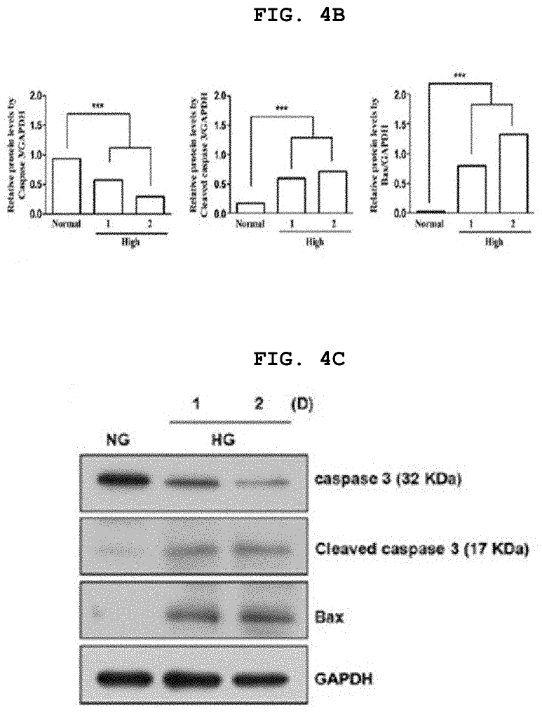

As a result, as shown in FIG. 4A to FIG. 4C, it was observed that the incubation at high glucose resulted in increased apoptosis, as measured by the increase in Bax and cleaved caspase-3. These results confirmed that the expression of TSPAN2 was induced by high glucose on the transcriptional level. In addition, considering that apoptosis was increased by high glucose, it was verified that increased expression of TSPAN2 affects apoptosis.

### Example 3: Effect of TSPAN2 Expression on Bax Expression

The cell signaling mechanism in which the expression of TSPAN2 is increased by high glucose was examined. The effect of the increased level of TSPAN2 on cell signaling pathway was investigated.

RNAKT-15 cells were incubated under normal glucose (NG) or high glucose (HG) conditions for 2 d, while the cells were transfected with plasmids overexpressing TSPAN2. The cell lysates were subjected to Western blot analysis.

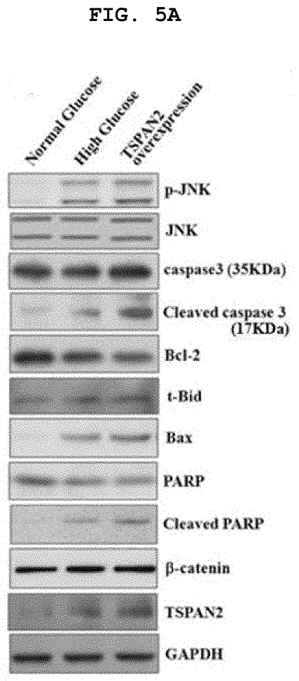

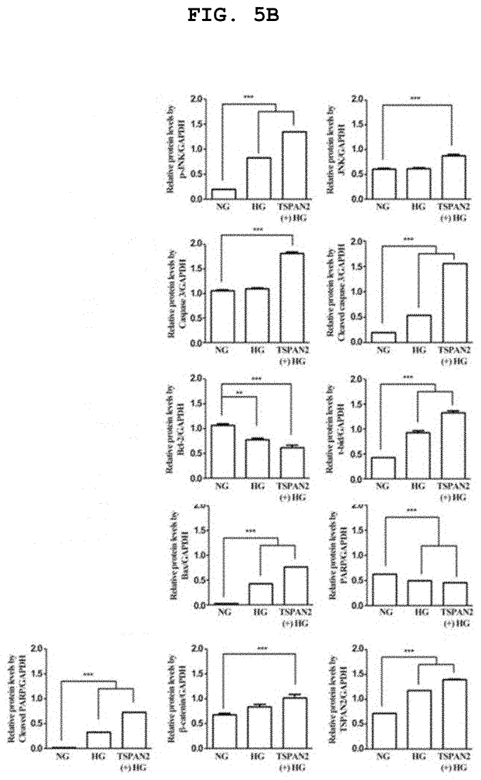

As a result, as shown in FIG. 5A and FIG. 5B, it was observed that p-JNK, Bax, t-Bid, and cleaved PARP were increased under high glucose. Also, it was observed that p-JNK, Bax, t-Bid, and cleaved PARP were increased in the same manner under normal glucose with the overexpression of TSPAN2. While, under high glucose and normal glucose conditions, the overexpression of TSPAN2 reduced the expression of Bcl-2 and PARP. These results indicate that high glucose induces glucose toxicity-induced apoptosis, while the overexpression of TSPAN2 shows similar effects.

In addition, to investigate the effect of TSPAN2 overexpression on glucose toxicity-induced apoptosis, TSPAN2 expression was silenced using TSPAN2 siRNA and the expression levels of Bax were determined.

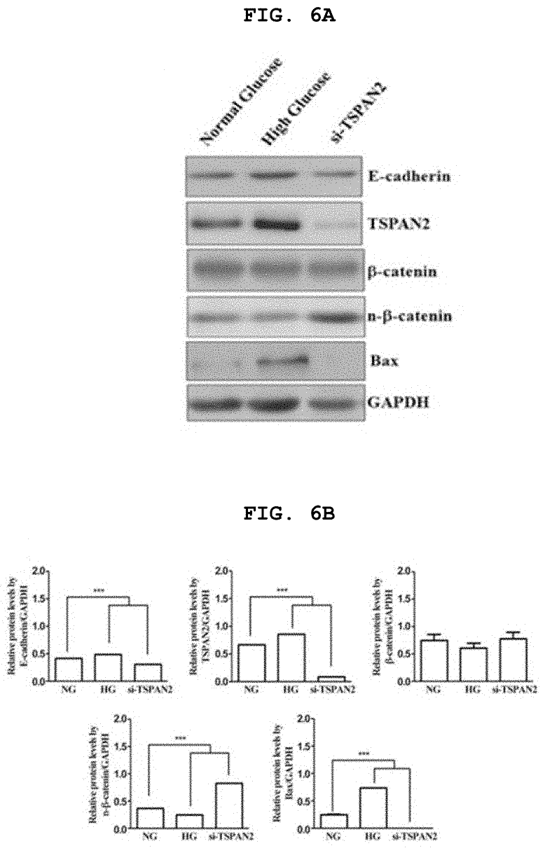

As a result, as shown in FIG. 6A and FIG. 6B, TSPAN2 siRNA efficiently silenced the expression of TSPAN protein. Although high glucose increased the expression level of Bax protein, TSPAN2 decreased the expression of Bax. These results suggest that the expression of TSPAN2 contributes to the reduction in the expression Bax under high glucose conditions. Furthermore, it was found that nuclear β-catenin was increased by TSPAN2 silencing.

### Example 4: Effect of JNK on β-Catenin

The potential role of JNK in the regulation of β-catenin signaling was examined.

RNAKT-15 cells were incubated, transfected with JNK and DKK siRNAs, and treated with JNK inhibitor SP600125. RNAKT-15 cells incubated in 12-well plate were incubated with β-catenin antibody, stained with DAPI, and observed by confocal microscopy. In addition, RNAKT-15 cells were incubated in normal glucose (NG) or high glucose (HG) conditions, respectively, for 2 d, and the obtained cells were subjected to Western blot.

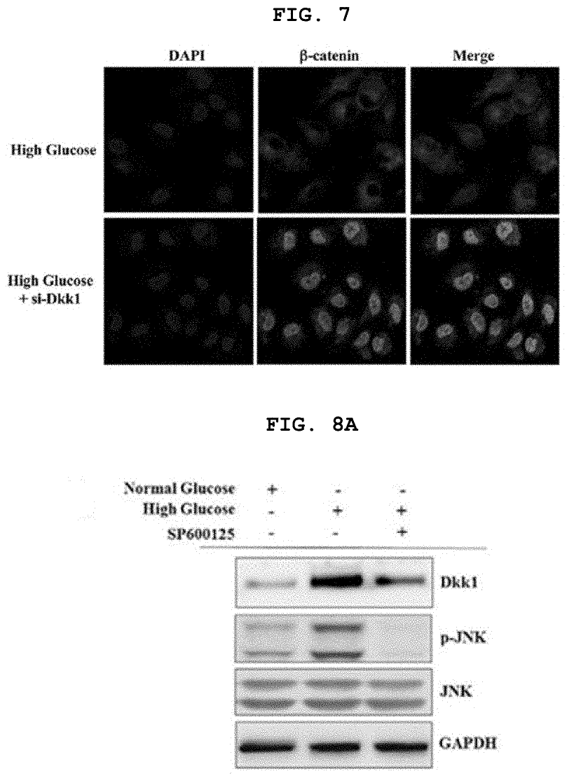

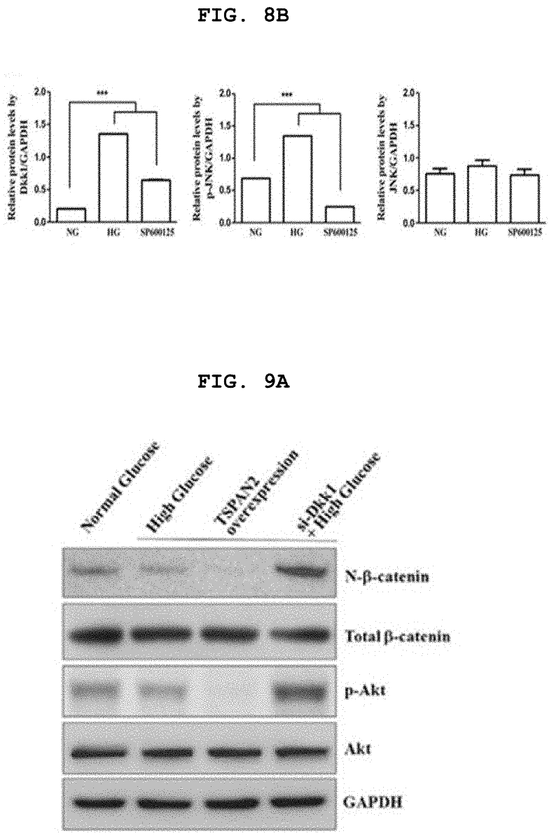

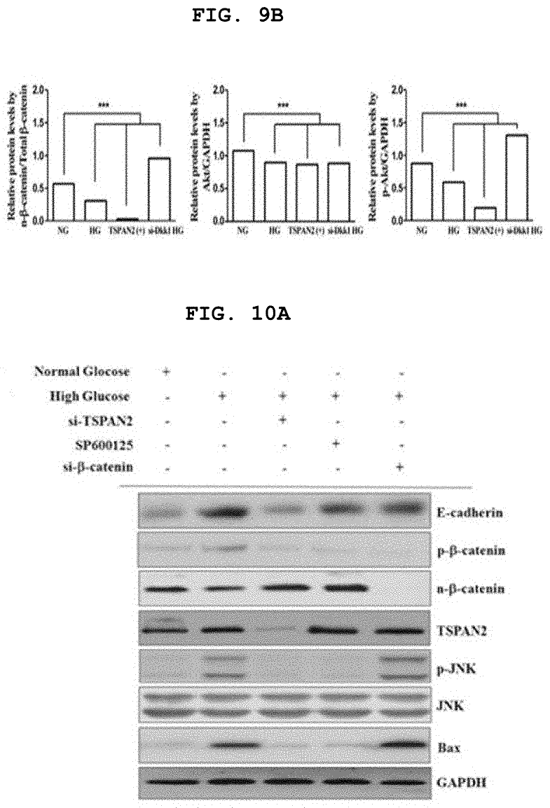

As a result, as shown in FIG. 8A, FIG. 8B, FIG. 9A and FIG. 9B, it was observed that, in the RNAKT-15 cells treated with JNK inhibitor (SP600125), the expression levels of Dkk1 were reduced, while the treatment with Dkk1 siRNA under HG conditions increased the expression levels of β-catenin and p-Akt. Similarly, β-catenin nuclear translocation was inhibited under HG, whereas both cytosolic and nuclear β-catenins were increased in the cells treated with Dkk1 siRNA (FIG. 7). In addition, HG-induced p-JNK up-regulated p-β-catenin and Dkk1, and si-Dkk1 promoted the nuclear translocation of β-catenin (FIG. 7). These results show that TSPAN2 effectively attenuates nuclear β-catenin translocation by regulating the JNK-Dkk1 signaling pathway.

### Example 5: Mechanism of TSPAN2 Activating Bax Through JNK

To examine whether TSPAN2 is linked functionally to JNK signaling pathway, changes in JNK signaling pathway was investigated in HG conditions.

It was observed that p-JNK was increased in HG conditions (FIG. 8A and FIG. 8B).

Then, a loss-of-function analysis was conducted using TSPAN2 knockdown by siRNA.

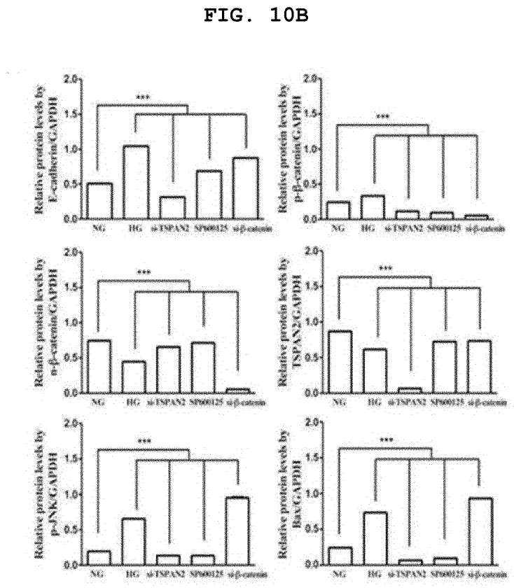

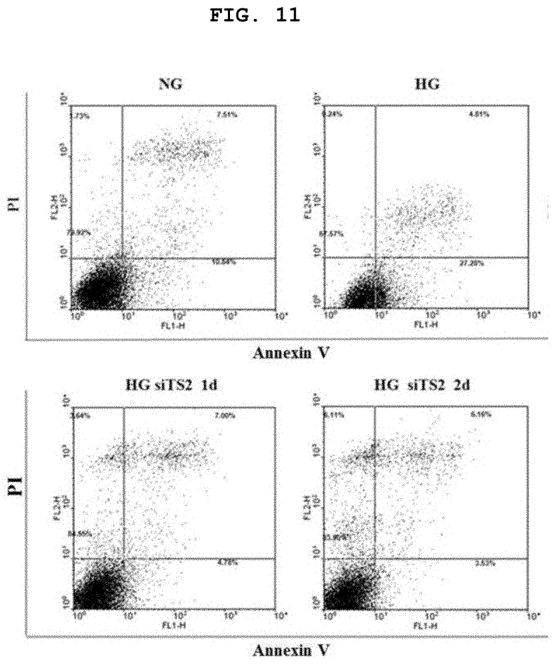

TSPAN2 siRNA suppressed p-JNK, and the selective JNK inhibitor (SP600125) blocked JNK and Bax activity. Immunoblots confirmed the reduction in p-JNK protein in RNAKT-15 cells, and TSPAN2 overexpression further enhanced the p-JNK protein-mediated increase in Bax (FIG. 10A and FIG. 10B). Furthermore, β-catenin siRNA significantly decreased nuclear β-catenin and increased Bax (FIG. 10A and FIG. 10B), indicating that β-catenin signaling promotes pancreatic β-cell survival by inhibiting Bax in RNAKT-15 cells. Taken together, these results show that TSPAN2 overexpression up-regulated the p-JNK levels and p-JNK-alleviated nuclear β-catenin translocation, leading to the up-regulating of Bax.

### Example 6: TSPAN2 and JNK/β-Catenin Signaling Pathway

Previous results suggested that the phosphorylation of JNK was increased under HG conditions, indicating that HG activates JNK. Because TSPAN2 expression is increased under HG conditions, the effect of JNK on Bax expression was examined.

Cells were either mock treated or transfected with TSPAN2 siRNA or β-catenin siRNA. Later, cells were treated with mock or a JNK inhibitor (SP600125), and the cell lysates were then subjected to Western blot analysis.

As a result, the silencing of TSPAN2 by siRNA or the SP600125 treatment inhibited JNK activation, and the expression of Bax decreased with similar patterns. These results suggest that JNK activation is involved in Bax expression. Furthermore, the level of nuclear β-catenin was increased by the silencing of TSPAN2 using siRAN or JNK inhibitor. These results indicate that the inhibition of JNK-mediated β-catenin signaling pathway activates Bax (FIG. 10A and FIG. 10B).

### Example 7: TSPAN2 Role in Glucose Toxicity-Induced Apoptosis

Because high glucose stimulation induced apoptosis in RNAKT-15 cells, the role of TSPAN2 in apoptosis was examined.

HG conditions significantly stimulated the expression of TSPAN2, resulting in excess production of p-JNK, which down-regulated nuclear β-catenin and its transcriptional activity. JNK-mediated β-catenin signaling inhibition increased the protein level of Bax by regulating the JNK/β-catenin pathway, which is inversely correlated with anti-apoptotic Bcl-2. Finally, the activation of caspase-3 further cleaves PARP in nuclei, leading to apoptosis of RNAKT-15 cells. Moreover, siRNA-mediated knockdown of TSPAN2, which inhibits the nuclear translocation of β-catenin, using siRNA, effectively prevented HG-induced apoptosis (FIG. 11). Therefore, TSPAN2 promotes the apoptosis in RNAKT-15 cells, and is significantly involved in glucose toxicity-induced apoptosis.

As described above, the composition and the screening method of the present invention can be favorably used in the development of a completely new mechanism of diabetes therapeutic agent that inhibits the expression or activity of TSPAN2 protein by using the role of TSPAN2 that promotes apoptosis of pancreatic β-cells through JNK and β-catenin under hyperglycemic conditions.

### Example 8: Identifying the Efficacy of Anti-TSPAN2 Antibody for the Treatment of Diabetes

8-1. Antibody Preparation and Exosome Loading Thereof for In Vivo Delivery

anti-TSPAN2 antibody(IH00D) was produced by commercial antibody production service (AbClon, Korea) By using Human TSPAN2 protein as a antigen, 4 hybridoma cell lines producing monoclonal anti-TSPAN2 antibodies were prepared and each of the anti-TSPAN2 antibodies was sequenced to reveal its CDR sequence. One of the CDR sequences is shown in SEQ ID Nos. 90-95 (CDR-H1:SEQ ID No 90; CDR-H2:SEQ ID No 91; CDR-H3:SEQ ID No 92; CDR-L1:SEQ ID No 93; CDR-L2:SEQ ID No 94; CDR-L3:SEQ ID No 95)

To effectively deliver the anti-TSPAN2 antibody into the body, a TSPAN2 Ab-loaded exosome (antibody transfected exosome) was constructed. The detailed method is as follows.

First, in order to isolate BD-MSC(bone marrow-derived mesenchymal stem cells) from bone marrow, bone marrow cells obtained from female non-diabetic C57BL/6 mice were cultured on alpha-MEM (15% fbs) media for 8-12 days (Replace media every 3 days). Then, the bone marrow cells were cultured for 15 days using 5% FBS high DMEM, and MSCs were isolated. MSCs were incubated for at least 30 minutes at room temperature. When subcultured, MSCs reached approximately 80% confluency and actively proliferated, the ExoQuick kit (SBI bio) was applied to 10 ml of MSC culture media, and exosomes were isolated by centrifugation at 300×g for 2-3 minutes. Approximately 50-300 ug of exosomes were obtained and stored in 500 ul of sterile PBS.

10 ul Exo-Fect solution (exosome transfection solution/sbi system biosciences), 10 ul PBS solution, 50 ul purified exosome, and anti-TSPAN2 antibodies (20 ug) were put into a sterilized 1.5 ml tube and mixed 3 times repeatedly. The exosome transfection solution mixed with the antibody and exosomes was incubated in a shaker for 1 hour at 37° C. and immediately placed on a tube in ice. To this solution, 30 ul of ExoQuickTC reagent was added and mixed 6 times. The mixture was stabilized on ice or at 4° C. for 30 minutes. To obtain an antibody-loaded exosome, the mixture was centrifuged at 13,000-14,000 rpm for 3 minutes using a microfuge, then the supernatant was removed, and pellet was obtained. The obtained pellet was dissolved in 300 ul 1×PBS.

8-2. Identifying the Efficacy of Anti-TSPAN2 Antibody for Diabetes Treatment in Type 1 Diabetic Animal Model

seven-week-old Balb/c mouse (SPF mouse) were purchased from Samtako Bio Korea Co. Ltd (Osan, Korea). Experimental animals were fed with sterile feed and water, raised in a condition of less than 10 animals per each cage. The day and night cycle was 12 hours. Animal experiments were conducted at the animal breeding institute of Korea University, under the supervision of the Institutional Animal Care and Use Committee(IACUC). Animal experiment procedures were performed according to the approved protocol of Korea University IACUC. To induce type 1 diabetes, streptozotocin(STZ) was administered to the mice. The STZ was purchased from Sigma Chemicals Co. of St. Louis, Mo., USA. STZ was dissolved in cold 0.01 M citrate buffer (pH 4.5), and 150 mg/kg of STZ was intraperitoneally administered to mice once. From the day of STZ injection, blood glucose levels were measured daily. Blood was collected from the tail of the experimental animals. Specifically, the tail tip of the mouse was cut with surgical scissors to collect 10 ul of blood. Blood glucose levels were measured using Gluco Dr Top device (Model: AGM-4100) from allmedicus Co. Ltd. Hyperglycemia occurred in all mice at 1-week after a streptozotocin infusion of 150 mg/kg i.p., then, diabetic mice was injected with 25 ug TSPAN2 Ab (exosome loaded form, see Example 8-1), s.c., three times weekly. After 4 weeks, the animals were sacrificed and evaluated for pancreas and other organs in control group (STZ-induced diabetic mice) and experimental group (Administration of anti-TSPAN2 antibody to diabetic mice induced by STZ).

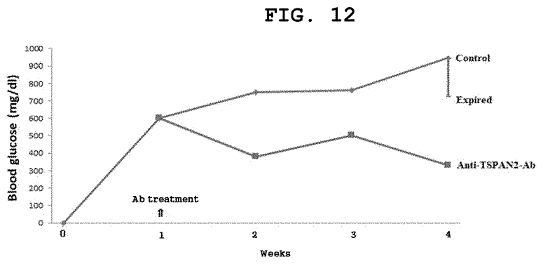

As a result, as shown in FIG. 12, the mice of the control group (type 1 diabetes mouse) maintained high blood glucose level, and all of them died after 4 weeks. On the other hand, in the mice of the experimental group injected with anti-TSPAN2 Ab, their blood glucose levels were significantly reduced and remained constant.

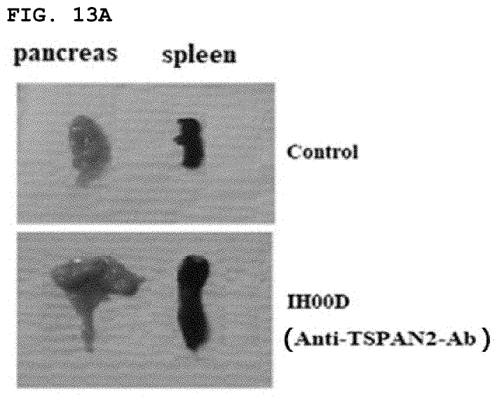

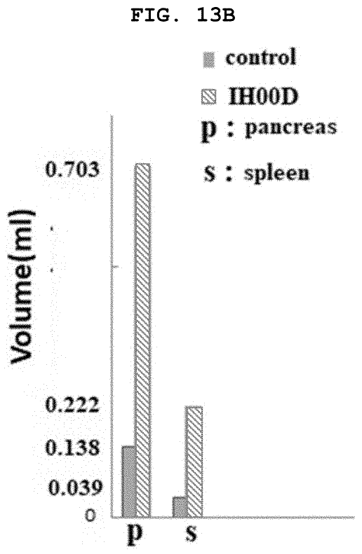

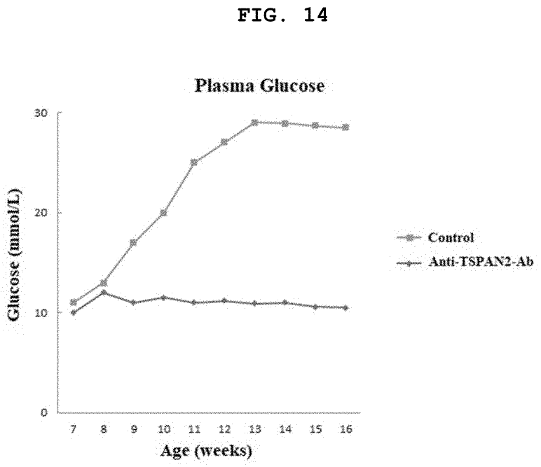

Also, as shown in FIG. 13A and FIG. 13B, in the mouse of control group (type 1 diabetes mouse), the mass and size of their pancreases and spleens were significantly decreased. On the other hand, in the experimental group injected with anti-TSPAN2 Ab into the pancreas, the mass and size of the pancreas and spleen were restored and maintained to normal. Based on these facts, it is confirmed that anti-TSPAN2 Ab administration showed a therapeutic effect through the protection of pancreatic beta cell function and the restoration of pancreatic cell etc., in type 1 diabetes mouse.

8-3. Identifying the Efficacy of Anti-TSPAN2 Antibody in the Treatment of Diabetes in Type 2 Diabetes

Seven-week-old db/db mice (type 2 diabetic mouse, SPF mouse), purchased from Samtako Bio Korea Co. Ltd (Osan, Korea). They were divided into groups of eight mice per group. Experimental animals were fed with sterile feed and water. The day and night cycle was 12 hours. Animal experiments were conducted at the animal breeding institute of Korea University, under the supervision of the Institutional Animal Care and Use Committee(IACUC). Animal experiment procedures were performed according to the approved protocol of Korea University IACUC. 100 ug of TSPAN2 Ab-loaded exosomes (an amount corresponding to 20 ug of anti-TSPAN2 Ab) were injected intrapancreatically to the mice, two times weekly. Blood glucose level was measured daily. Blood was collected from the tail of each db/db mouse and glucose concentration was measured, in the same manner as in Example 8-2.

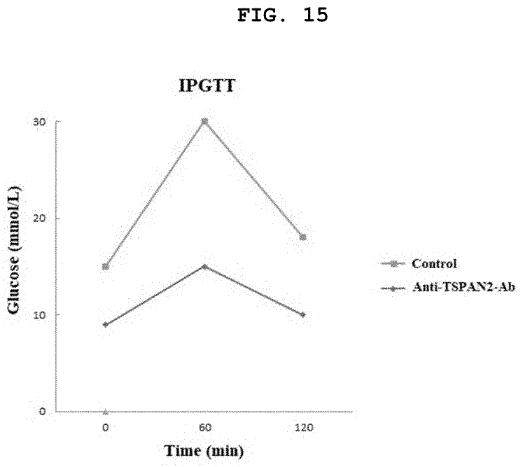

As a result, as shown in FIG. 14, in the db/db mice (type 2 diabetes model), blood glucose level was continuously increased. On the other hand, in db/db mice administered with anti-TSPAN2 Ab, it was confirmed that blood glucose level remained constant at a significantly low level. Thus, it was confirmed that anti-TSPAN2 Ab administration had an excellent blood glucose lowering effect, suggesting that anti-TSPAN2 Ab administration has a therapeutic effect of type 2 diabetes.

Also, The effect of anti-TSPAN2 Ab administration on glucose tolerance and insulin sensitivity was confirmed. For this experiment, Seven-week-old db/db mice (type 2 diabetic mouse) were divided into groups of eight mice per group. 100 ug of TSPAN2 Ab-loaded exosomes (an amount corresponding to 20 ug of anti-TSPAN2 Ab) was injected intra-pancreatically to the mice, two times weekly. Then, the glucose tolerance and insulin sensitivity of db/db mice were measured.

Specifically, when db/db mice becomes 10 weeks old, IPGTT(intraperitoneal blood glucose tolerance test) was performed by intraperitoneal administration of glucose (2 g per kg body weight of the experimental animals). Glucose levels were tested in the same manner as in Example 8-2, and monitored at 30 min, 60 min, and 120 min after glucose administration.

When db/db mice becomes 14 weeks old, ITT(Insulin Tolerance Test) was performed by intraperitoneal injection of insulin (0.5 U per kg body weight of the experimental animal). Glucose levels were monitored by tail-bleeding assay method (in the same manner as in Example 8-2) and monitored at 10 min, 20 min, 40 min, and 60 min after insulin administration.

As shown in FIG. 15, as a result of measuring the change of blood glucose level with time after glucose administration, it was confirmed that blood glucose level was significantly lowered in the anti-TSPAN2 Ab-treated group compared to the db/db mouse control group (type 2 diabetic mouse).

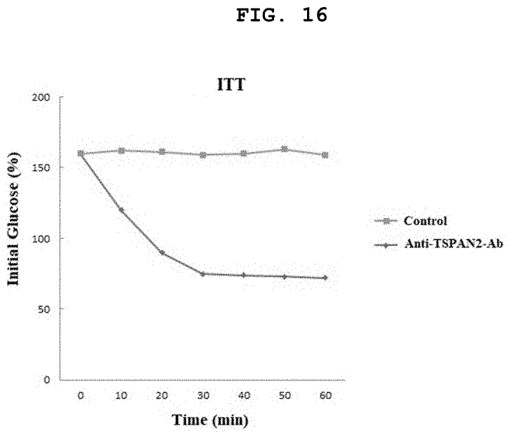

Also, as shown in FIG. 16, as a result of measuring the change of blood glucose level over time after insulin administration, it was confirmed that blood glucose was rapidly removed in the test group administered with anti-TSPAN2 Ab as compared with the db/db mouse control group (type 2 diabetic mouse). These results show that the anti-TSPAN2 Ab administration significantly improves the glucose tolerance and insulin sensitivity, suggesting its therapeutic effect of type 2 diabetes.

Through the results of Example 8 above, the inhibition of TSPAN2 is applicable in the treatment of all type of diabetes including Type 1 diabetes mellitus, Type 2 diabetes mellitus, and gestational diabetes mellitus etc.

What is claimed is:

1. A method for treating diabetes in a subject in need thereof, the method comprising administering to the subject a composition comprising a tetraspanin-2 (TSPAN2) protein activity inhibitor as an active ingredient in an amount effective for treating diabetes.

2. The method of claim 1, wherein the diabetes is selected from the group consisting of Type 1 diabetes mellitus, Type 2 diabetes mellitus, and gestational diabetes mellitus.

3. The method of claim 1, wherein the TSPAN2 protein includes the amino acid sequence represented by any one sequence selected from the group consisting of SEQ ID NO: 1 to SEQ ID NO: 3.

4. The method of claim 1, wherein the TSPAN2 protein activity inhibitor is any one selected from the group consisting of a compound, a peptide, a peptide mimetic, an aptamer, an antibody, a natural extract, and a synthetic compound, each of which specifically binds to the TSPAN2 protein.

5. The method of claim 4, wherein the TSPAN2 protein activity inhibitor is an antibody specifically binding to the TSPAN2 protein.

6. The method of claim 5, wherein the antibody is provided in form of antibody-loaded exosome.


SEQUENCE LISTING (95 sequences)

SEQ ID NO: 1 - protein, 221 aa, Artificial Sequence
  Homo sapiens tetraspanin-2 isoform 1 (NP_005716.2)
MGRFRGGLRCIKYLLLGFNLLFWLAGSAVIAFGLWFRFGGAIKELSSEDKSPEYFYVGLYVLVGAGALMMAVGFFGCCGAMRESQCVLGSFFTCLLVIFAAEVTTGVFAFIGKGVAIRHVQTMYEEAYNDYLKDRGKGNGTLITFHSTFQCCGKESSEQVQPTCPKELLGHKNCIDEIETIISVKLQLIGIVGIGIAGLTIFGMIFSMVLCCAIRNSRDVI

SEQ ID NO: 2 - protein, 196 aa, Artificial Sequence
  Homo sapiens tetraspanin-2 isoform 2 (NP_001295244.1)
MGRFRGGLRCIKYLLLGFNLLFWLAGSAVIAFGLWFRFGGAIKELSSEDKSPEYFYVGLYVLVGAGALMMAVGFFGCCGAMRESQCVLGSAIRHVQTMYEEAYNDYLKDRGKGNGTLITFHSTFQCCGKESSEQVQPTCPKELLGHKNCIDEIETIISVKLQLIGIVGIGIAGLTIFGMIFSMVLCCAIRNSRDVI

SEQ ID NO: 3 - protein, 193 aa, Artificial Sequence
  Homo sapiens tetraspanin-2 isoform 3 (NP_001295245.1)
MGRFRGGLRCIKYLLLGFNLLFWLAGSAVIAFGLWFRFGGAIKELSSEDKSPEYFYVGLYVLVGAGALMMAVGFFGCCGAMRESQCVLGSFFTCLLVIFAAEVTTGVFAFIGKGVAIRHVQTMYEEAYNDYLKDRGKGNGTLITFHSTFQCCGKESSEQVQPTCPKELLGHKIFGMIFSMVLCCAIRNSRDVI

SEQ ID NO: 4 - DNA, 3227 bp, Artificial Sequence
  Homo sapiens tetraspanin-2 mRNA transcript variant 1 (NM_005725.5)
ggagcctgccgctccccgcgctcgtagcgcgggcctggggactggggatcccgccgccgggccgcagcatggggcgcttccgcgggggcctgcggtgcatcaagtacctgctgcttggcttcaacctgctcttctggctggctggatcggccgtcattgcttttggactatggtttcggttcggaggtgccataaaggagttatcatcagaggacaagtccccagagtatttctatgtggggctgtatgttctggttggagccggggccctgatgatggccgtggggttcttcgggtgctgcggagccatgcgggagtcgcaatgtgtgcttggatcattttttacctgcctcctggtgatatttgctgctgaagtaaccactggagtatttgcttttataggcaagggggtagctatccgacatgttcagaccatgtatgaagaggcttacaatgattaccttaaagacaggggaaaaggcaatgggacactcatcaccttccactcaacatttcagtgctgtggaaaagaaagctccgaacaggtccaacctacatgcccaaaggagcttctaggacacaagaattgcatcgatgaaattgagaccataatcagtgttaagctccagctcattggaattgtcggtattggaattgcaggtctgacgatctttggcatgatattcagcatggtcctctgctgtgcgatacgaaactcacgagatgtgatatgaagctacttctacatgaaaattgcaatctaaagctttcataccaaatgtcacaggagctgtctcccagctcatttttaacactgaaatgacattaggatctaaaataatttgctgtcaattgtacatttgcatgagtacgtatgtttggctcattactggtttaccccttgagtgaatgcctgtttatgatgactgagagcatattcatgtgtgatctgcgtgtttctggaatatgctttatacgtaatgaaatctgtttgctgggaattcctgattcttgttatataagaagaacaacctatttcgctcccagaaaaaaaagatcaaagagctttcagaaactttgagaacttggctatttagaaaaagtgataatgggtcagtttctcagactgtagccattgaaaattagatgcagagaattcagagatttcttcttaatggaagtaataagctgtaagaattgagagatcacaatggagtgttaaaactgactgtgtctaagttgggtgtaagggtttcctgggtttttttatatacatgctctccccagaatacagtaaaccacagttttagaactaaacacatctgtaaaactaaatatagcatggaaaatccaatttgaataagtcatgctttcctagaatttaaaaataaaaaagtcttcctctggaaagagaagtcacacagacaatcatgtgccctataaaagtgagtgtttataggactaaaaaacttttaacaactttttaaggaaatatttttgttcttatacaaaaacatgtaaatattgctttattactttcattttctgaccctgctgtaaactactgcaaccctcacatcctcaaagggacttttatgtcaaactcttctgtttctccaaatataaggaaaaaagactaaagcaagagatctggcagttgaaaattgtgggaaagagaatttgtatgggcactgtatctatgaaatacctcataacttacgtttacatgttttcctaactttttgtatttttcttgtatagccacctagagaattcttcatagattaagaactacagttttcaccacttaacataagtaaaacaaagtccttcataatttaaccattagcatctttggccaaaccaaaataaagaaaagcatcttctcctagttgtgtgtgggcaacagaaacaagttaaggaaacaaaaatacttatatatacacagaacaaaaataatgttctttttatgcaaatcccctgtgaaaataaaattttcaatgtttttttggtcttaacgcttcattttaacttagtaaacccaaatttaggatttacattggcaaatttttcagtgatttttaacgtgttacatttagtaaaccatagattttttaaaaactacattttgtaaggccacacataagtgaaaatacggaaggctttggaaacccccagatgaacttctatacatgtgaaaacacaagtctttgagtgtgcactaaaagtgtcacactcccaaaaaactcacaaaacattttgctaaaaagatatgactgcttgaaatagtctctctcccatggagaaccacatttctctttctgccatacttgctactgctactgattttggaggaaatttttctagaagaacaaaggtcatatttgaggcaagctaaaaacttaattatacatggctactttactgtgtctgtagctaaagaaaaattattttacaaaaattacttcatgaaatatagtttgtaccccagtagttgagaaatgtgaccttttaggcaaagaacaggcagtccatctctcttagctttagtttatctctctgcctgaagacaggaaacaggcccaagtgcatactcgggttctttccaactcagaatcatctctgattccacaaaagtgagtttagtttcctatctgaattaacaactttaaaggagactataatagttaaaagtggaagaatagaaataaataaatttaaaatgaaattaattaaagtagaagagaagggttctgttccatgtacgattaatgtgccccttttggagataagctttccaaaatatgcgtatgagtaaaattagagaattccaccactaagcaacaatagattttatatgtgtgtgtggatatatatacacacacacacagaaagatgaatatatatatatatatatctgtatagacacacatataatatatatagatatataatccctatatatcttgaatctacaaatatctttatatgtatataatttgaatatatacatgtatgtatatacagatgtgtgtgtatatatatatatacatacacatatacataaattttcagctatttctgttcccctgaactaatttgagattagacccaaacatgcttttaaatgtctcatgccaaggcactgaattaagacttcttaatgttgaaatattttaagtgatttcatattaaattttctatatactgaaaaaaaaaaaaaaaaa

SEQ ID NO: 5 - DNA, 3152 bp, Artificial Sequence
  Homo sapiens tetraspanin-2 mRNA transcript variant 2 (NM_001308315.1)
ggagcctgccgctccccgcgctcgtagcgcgggcctggggactggggatcccgccgccgggccgcagcatggggcgcttccgcgggggcctgcggtgcatcaagtacctgctgcttggcttcaacctgctcttctggctggctggatcggccgtcattgcttttggactatggtttcggttcggaggtgccataaaggagttatcatcagaggacaagtccccagagtatttctatgtggggctgtatgttctggttggagccggggccctgatgatggccgtggggttcttcgggtgctgcggagccatgcgggagtcgcaatgtgtgcttggatcagctatccgacatgttcagaccatgtatgaagaggcttacaatgattaccttaaagacaggggaaaaggcaatgggacactcatcaccttccactcaacatttcagtgctgtggaaaagaaagctccgaacaggtccaacctacatgcccaaaggagcttctaggacacaagaattgcatcgatgaaattgagaccataatcagtgttaagctccagctcattggaattgtcggtattggaattgcaggtctgacgatctttggcatgatattcagcatggtcctctgctgtgcgatacgaaactcacgagatgtgatatgaagctacttctacatgaaaattgcaatctaaagctttcataccaaatgtcacaggagctgtctcccagctcatttttaacactgaaatgacattaggatctaaaataatttgctgtcaattgtacatttgcatgagtacgtatgtttggctcattactggtttaccccttgagtgaatgcctgtttatgatgactgagagcatattcatgtgtgatctgcgtgtttctggaatatgctttatacgtaatgaaatctgtttgctgggaattcctgattcttgttatataagaagaacaacctatttcgctcccagaaaaaaaagatcaaagagctttcagaaactttgagaacttggctatttagaaaaagtgataatgggtcagtttctcagactgtagccattgaaaattagatgcagagaattcagagatttcttcttaatggaagtaataagctgtaagaattgagagatcacaatggagtgttaaaactgactgtgtctaagttgggtgtaagggtttcctgggtttttttatatacatgctctccccagaatacagtaaaccacagttttagaactaaacacatctgtaaaactaaatatagcatggaaaatccaatttgaataagtcatgctttcctagaatttaaaaataaaaaagtcttcctctggaaagagaagtcacacagacaatcatgtgccctataaaagtgagtgtttataggactaaaaaacttttaacaactttttaaggaaatatttttgttcttatacaaaaacatgtaaatattgctttattactttcattttctgaccctgctgtaaactactgcaaccctcacatcctcaaagggacttttatgtcaaactcttctgtttctccaaatataaggaaaaaagactaaagcaagagatctggcagttgaaaattgtgggaaagagaatttgtatgggcactgtatctatgaaatacctcataacttacgtttacatgttttcctaactttttgtatttttcttgtatagccacctagagaattcttcatagattaagaactacagttttcaccacttaacataagtaaaacaaagtccttcataatttaaccattagcatctttggccaaaccaaaataaagaaaagcatcttctcctagttgtgtgtgggcaacagaaacaagttaaggaaacaaaaatacttatatatacacagaacaaaaataatgttctttttatgcaaatcccctgtgaaaataaaattttcaatgtttttttggtcttaacgcttcattttaacttagtaaacccaaatttaggatttacattggcaaatttttcagtgatttttaacgtgttacatttagtaaaccatagattttttaaaaactacattttgtaaggccacacataagtgaaaatacggaaggctttggaaacccccagatgaacttctatacatgtgaaaacacaagtctttgagtgtgcactaaaagtgtcacactcccaaaaaactcacaaaacattttgctaaaaagatatgactgcttgaaatagtctctctcccatggagaaccacatttctctttctgccatacttgctactgctactgattttggaggaaatttttctagaagaacaaaggtcatatttgaggcaagctaaaaacttaattatacatggctactttactgtgtctgtagctaaagaaaaattattttacaaaaattacttcatgaaatatagtttgtaccccagtagttgagaaatgtgaccttttaggcaaagaacaggcagtccatctctcttagctttagtttatctctctgcctgaagacaggaaacaggcccaagtgcatactcgggttctttccaactcagaatcatctctgattccacaaaagtgagtttagtttcctatctgaattaacaactttaaaggagactataatagttaaaagtggaagaatagaaataaataaatttaaaatgaaattaattaaagtagaagagaagggttctgttccatgtacgattaatgtgccccttttggagataagctttccaaaatatgcgtatgagtaaaattagagaattccaccactaagcaacaatagattttatatgtgtgtgtggatatatatacacacacacacagaaagatgaatatatatatatatatatctgtatagacacacatataatatatatagatatataatccctatatatcttgaatctacaaatatctttatatgtatataatttgaatatatacatgtatgtatatacagatgtgtgtgtatatatatatatacatacacatatacataaattttcagctatttctgttcccctgaactaatttgagattagacccaaacatgcttttaaatgtctcatgccaaggcactgaattaagacttcttaatgttgaaatattttaagtgatttcatattaaattttctatatactgaaaaaaaaaaaaaaaaa

SEQ ID NO: 6 - DNA, 3143 bp, Artificial Sequence
  Homo sapiens tetraspanin-2 mRNA transcript variant 3 (NM_001308316.1)
ggagcctgccgctccccgcgctcgtagcgcgggcctggggactggggatcccgccgccgggccgcagcatggggcgcttccgcgggggcctgcggtgcatcaagtacctgctgcttggcttcaacctgctcttctggctggctggatcggccgtcattgcttttggactatggtttcggttcggaggtgccataaaggagttatcatcagaggacaagtccccagagtatttctatgtggggctgtatgttctggttggagccggggccctgatgatggccgtggggttcttcgggtgctgcggagccatgcgggagtcgcaatgtgtgcttggatcattttttacctgcctcctggtgatatttgctgctgaagtaaccactggagtatttgcttttataggcaagggggtagctatccgacatgttcagaccatgtatgaagaggcttacaatgattaccttaaagacaggggaaaaggcaatgggacactcatcaccttccactcaacatttcagtgctgtggaaaagaaagctccgaacaggtccaacctacatgcccaaaggagcttctaggacacaagatctttggcatgatattcagcatggtcctctgctgtgcgatacgaaactcacgagatgtgatatgaagctacttctacatgaaaattgcaatctaaagctttcataccaaatgtcacaggagctgtctcccagctcatttttaacactgaaatgacattaggatctaaaataatttgctgtcaattgtacatttgcatgagtacgtatgtttggctcattactggtttaccccttgagtgaatgcctgtttatgatgactgagagcatattcatgtgtgatctgcgtgtttctggaatatgctttatacgtaatgaaatctgtttgctgggaattcctgattcttgttatataagaagaacaacctatttcgctcccagaaaaaaaagatcaaagagctttcagaaactttgagaacttggctatttagaaaaagtgataatgggtcagtttctcagactgtagccattgaaaattagatgcagagaattcagagatttcttcttaatggaagtaataagctgtaagaattgagagatcacaatggagtgttaaaactgactgtgtctaagttgggtgtaagggtttcctgggtttttttatatacatgctctccccagaatacagtaaaccacagttttagaactaaacacatctgtaaaactaaatatagcatggaaaatccaatttgaataagtcatgctttcctagaatttaaaaataaaaaagtcttcctctggaaagagaagtcacacagacaatcatgtgccctataaaagtgagtgtttataggactaaaaaacttttaacaactttttaaggaaatatttttgttcttatacaaaaacatgtaaatattgctttattactttcattttctgaccctgctgtaaactactgcaaccctcacatcctcaaagggacttttatgtcaaactcttctgtttctccaaatataaggaaaaaagactaaagcaagagatctggcagttgaaaattgtgggaaagagaatttgtatgggcactgtatctatgaaatacctcataacttacgtttacatgttttcctaactttttgtatttttcttgtatagccacctagagaattcttcatagattaagaactacagttttcaccacttaacataagtaaaacaaagtccttcataatttaaccattagcatctttggccaaaccaaaataaagaaaagcatcttctcctagttgtgtgtgggcaacagaaacaagttaaggaaacaaaaatacttatatatacacagaacaaaaataatgttctttttatgcaaatcccctgtgaaaataaaattttcaatgtttttttggtcttaacgcttcattttaacttagtaaacccaaatttaggatttacattggcaaatttttcagtgatttttaacgtgttacatttagtaaaccatagattttttaaaaactacattttgtaaggccacacataagtgaaaatacggaaggctttggaaacccccagatgaacttctatacatgtgaaaacacaagtctttgagtgtgcactaaaagtgtcacactcccaaaaaactcacaaaacattttgctaaaaagatatgactgcttgaaatagtctctctcccatggagaaccacatttctctttctgccatacttgctactgctactgattttggaggaaatttttctagaagaacaaaggtcatatttgaggcaagctaaaaacttaattatacatggctactttactgtgtctgtagctaaagaaaaattattttacaaaaattacttcatgaaatatagtttgtaccccagtagttgagaaatgtgaccttttaggcaaagaacaggcagtccatctctcttagctttagtttatctctctgcctgaagacaggaaacaggcccaagtgcatactcgggttctttccaactcagaatcatctctgattccacaaaagtgagtttagtttcctatctgaattaacaactttaaaggagactataatagttaaaagtggaagaatagaaataaataaatttaaaatgaaattaattaaagtagaagagaagggttctgttccatgtacgattaatgtgccccttttggagataagctttccaaaatatgcgtatgagtaaaattagagaattccaccactaagcaacaatagattttatatgtgtgtgtggatatatatacacacacacacagaaagatgaatatatatatatatatatctgtatagacacacatataatatatatagatatataatccctatatatcttgaatctacaaatatctttatatgtatataatttgaatatatacatgtatgtatatacagatgtgtgtgtatatatatatatacatacacatatacataaattttcagctatttctgttcccctgaactaatttgagattagacccaaacatgcttttaaatgtctcatgccaaggcactgaattaagacttcttaatgttgaaatattttaagtgatttcatattaaattttctatatactgaaaaaaaaaaaaaaaaa

SEQ ID NO: 7 - DNA, 21 bp, Artificial Sequence
  siRNA#1 for tetraspanin-2 mRNA, transcript variant 1 (NM_005725.5)
aagctttcataccaaatgtca

SEQ ID NO: 8 - DNA, 21 bp, Artificial Sequence
  siRNA#2 for tetraspanin-2 mRNA, transcript variant 1 (NM_005725.5)
aagaacaggcagtccatctct

SEQ ID NO: 9 - DNA, 21 bp, Artificial Sequence
  siRNA#3 for tetraspanin-2 mRNA, transcript variant 1 (NM_005725.5)
aagtcttcctctggaaagaga

SEQ ID NO: 10 - DNA, 21 bp, Artificial Sequence
  siRNA#4 for tetraspanin-2 mRNA, transcript variant 1 (NM_005725.5)
aagcaagagatctggcagttg

SEQ ID NO: 11 - DNA, 21 bp, Artificial Sequence
  siRNA#5 for tetraspanin-2 mRNA, transcript variant 1 (NM_005725.5)
aaggagttatcatcagaggac

SEQ ID NO: 12 - DNA, 21 bp, Artificial Sequence
  siRNA#6 for tetraspanin-2 mRNA, transcript variant 1 (NM_005725.5)
aagagctttcagaaactttga

SEQ ID NO: 13 - DNA, 21 bp, Artificial Sequence
  siRNA#7 for tetraspanin-2 mRNA, transcript variant 1 (NM_005725.5)
aagaggcttacaatgattacc

SEQ ID NO: 14 - DNA, 21 bp, Artificial Sequence
  siRNA#8 for tetraspanin-2 mRNA, transcript variant 1 (NM_005725.5)
aagtctttgagtgtgcactaa

SEQ ID NO: 15 - DNA, 21 bp, Artificial Sequence
  siRNA#9 for tetraspanin-2 mRNA, transcript variant 1 (NM_005725.5)
aaggcactgaattaagacttc

SEQ ID NO: 16 - DNA, 21 bp, Artificial Sequence
  siRNA#1 for tetraspanin-2 mRNA, transcript variant 2 (NM_001308315.1)
aagctttcataccaaatgtca

SEQ ID NO: 17 - DNA, 21 bp, Artificial Sequence
  siRNA#2 for tetraspanin-2 mRNA, transcript variant 2 (NM_001308315.1)
aagaacaggcagtccatctct

SEQ ID NO: 18 - DNA, 21 bp, Artificial Sequence
  siRNA#3 for tetraspanin-2 mRNA, transcript variant 2 (NM_001308315.1)
aagtcttcctctggaaagaga

SEQ ID NO: 19 - DNA, 21 bp, Artificial Sequence
  siRNA#4 for tetraspanin-2 mRNA, transcript variant 2 (NM_001308315.1)
aagcaagagatctggcagttg

SEQ ID NO: 20 - DNA, 21 bp, Artificial Sequence
  siRNA#5 for tetraspanin-2 mRNA, transcript variant 2 (NM_001308315.1)
aaggagttatcatcagaggac

SEQ ID NO: 21 - DNA, 21 bp, Artificial Sequence
  siRNA#6 for tetraspanin-2 mRNA, transcript variant 2 (NM_001308315.1)
aagagctttcagaaactttga

SEQ ID NO: 22 - DNA, 21 bp, Artificial Sequence
  siRNA#7 for tetraspanin-2 mRNA, transcript variant 2 (NM_001308315.1)
aagaggcttacaatgattacc

SEQ ID NO: 23 - DNA, 21 bp, Artificial Sequence
  siRNA#8 for tetraspanin-2 mRNA, transcript variant 2 (NM_001308315.1)
aagtctttgagtgtgcactaa

SEQ ID NO: 24 - DNA, 21 bp, Artificial Sequence
  siRNA#9 for tetraspanin-2 mRNA, transcript variant 2 (NM_001308315.1)
aaggcactgaattaagacttc

SEQ ID NO: 25 - DNA, 21 bp, Artificial Sequence
  siRNA#1 for tetraspanin-2 mRNA, transcript variant 3 (NM_001308316.1)
aagctttcataccaaatgtca

SEQ ID NO: 26 - DNA, 21 bp, Artificial Sequence
  siRNA#2 for tetraspanin-2 mRNA, transcript variant 3 (NM_001308316.1)
aagaacaggcagtccatctct

SEQ ID NO: 27 - DNA, 21 bp, Artificial Sequence
  siRNA#3 for tetraspanin-2 mRNA, transcript variant 3 (NM_001308316.1)
aagtcttcctctggaaagaga

SEQ ID NO: 28 - DNA, 21 bp, Artificial Sequence
  siRNA#4 for tetraspanin-2 mRNA, transcript variant 3 (NM_001308316.1)
aagcaagagatctggcagttg

SEQ ID NO: 29 - DNA, 21 bp, Artificial Sequence
  siRNA#5 for tetraspanin-2 mRNA, transcript variant 3 (NM_001308316.1)
aaggagttatcatcagaggac

SEQ ID NO: 30 - DNA, 21 bp, Artificial Sequence
  siRNA#6 for tetraspanin-2 mRNA, transcript variant 3 (NM_001308316.1)
aagagctttcagaaactttga

SEQ ID NO: 31 - DNA, 21 bp, Artificial Sequence
  siRNA#7 for tetraspanin-2 mRNA, transcript variant 3 (NM_001308316.1)
aagaggcttacaatgattacc

SEQ ID NO: 32 - DNA, 21 bp, Artificial Sequence
  siRNA#8 for tetraspanin-2 mRNA, transcript variant 3 (NM_001308316.1)
aagtctttgagtgtgcactaa

SEQ ID NO: 33 - DNA, 21 bp, Artificial Sequence
  siRNA#9 for tetraspanin-2 mRNA, transcript variant 3 (NM_001308316.1)
aaggcactgaattaagacttc

SEQ ID NO: 34 - DNA, 21 bp, Artificial Sequence
  DKK1 siRNA#1
aagaaccaccttgtcttcaaa

SEQ ID NO: 35 - DNA, 21 bp, Artificial Sequence
  DKK1 siRNA#2
aaggctctcatggactagaaa

SEQ ID NO: 36 - DNA, 21 bp, Artificial Sequence
  DKK1 siRNA#3
aagtaccagaccattgacaac

SEQ ID NO: 37 - DNA, 21 bp, Artificial Sequence
  DKK1 siRNA#4
aagaaggtcaagtgtgtacca

SEQ ID NO: 38 - DNA, 21 bp, Artificial Sequence
  DKK1 siRNA#5
aagcattccaataacaccttc

SEQ ID NO: 39 - DNA, 21 bp, Artificial Sequence
  DKK1 siRNA#6
aagccagtaattcttctaggc

SEQ ID NO: 40 - DNA, 21 bp, Artificial Sequence
  DKK1 siRNA#7
aagatcaccatcaagccagta

SEQ ID NO: 41 - DNA, 21 bp, Artificial Sequence
  DKK1 siRNA#8
aagtgtgtaccaagcatagga

SEQ ID NO: 42 - DNA, 21 bp, Artificial Sequence
  DKK1 siRNA#9
aaggatctcttggaatgacaa

SEQ ID NO: 43 - DNA, 21 bp, Artificial Sequence
  DKK1 siRNA#10
aaggttctgtttgtctccggt

SEQ ID NO: 44 - DNA, 20 bp, Artificial Sequence
  RT-PCR primer set 1 forward for TSPAN2 transcript variant 1(NM_005725.5)
ttggaattgtcggtattgga

SEQ ID NO: 45 - DNA, 20 bp, Artificial Sequence
  RT-PCR primer set 1 reverse for TSPAN2 transcript variant 1(NM_005725.5)
tctcgtgagtttcgtatcgc

SEQ ID NO: 46 - DNA, 20 bp, Artificial Sequence
  RT-PCR primer set 2 forward for TSPAN2 transcript variant 1(NM_005725.5)
aacctacatgcccaaaggag

SEQ ID NO: 47 - DNA, 20 bp, Artificial Sequence
  RT-PCR primer set 2 reverse for TSPAN2 transcript variant 1(NM_005725.5)
atcatgccaaagatcgtcag

SEQ ID NO: 48 - DNA, 20 bp, Artificial Sequence
  RT-PCR primer set 3 forward for TSPAN2 transcript variant 1(NM_005725.5)
ctgacgatctttggcatgat

SEQ ID NO: 49 - DNA, 20 bp, Artificial Sequence
  RT-PCR primer set 3 reverse for TSPAN2 transcript variant 1(NM_005725.5)
acagctcctgtgacatttgg

SEQ ID NO: 50 - DNA, 20 bp, Artificial Sequence
  RT-PCR primer set 4 forward for TSPAN2 transcript variant 1(NM_005725.5)
aagaaagctccgaacaggtc

SEQ ID NO: 51 - DNA, 20 bp, Artificial Sequence
  RT-PCR primer set 4 reverse for TSPAN2 transcript variant 1(NM_005725.5)
cgatgcaattcttgtgtcct

SEQ ID NO: 52 - DNA, 20 bp, Artificial Sequence
  RT-PCR primer set 5 forward for TSPAN2 transcript variant 1(NM_005725.5)
aaaggcaatgggacactcat

SEQ ID NO: 53 - DNA, 20 bp, Artificial Sequence
  RT-PCR primer set 5 reverse for TSPAN2 transcript variant 1(NM_005725.5)
gacctgttcggagctttctt

SEQ ID NO: 54 - DNA, 20 bp, Artificial Sequence
  RT-PCR primer set 6 forward for TSPAN2 transcript variant 1(NM_005725.5)
tcaacatttcagtgctgtgg

SEQ ID NO: 55 - DNA, 19 bp, Artificial Sequence
  RT-PCR primer set 6 reverse for TSPAN2 transcript variant 1(NM_005725.5)
aagctcctttgggcatgta

SEQ ID NO: 56 - DNA, 20 bp, Artificial Sequence
  RT-PCR primer set 1 forward for TSPAN2 transcript variant 2 (NM_001308315.1)
ttggaattgtcggtattgga

SEQ ID NO: 57 - DNA, 20 bp, Artificial Sequence
  RT-PCR primer set 1 reverse for TSPAN2 transcript variant 2 (NM_001308315.1)
tctcgtgagtttcgtatcgc

SEQ ID NO: 58 - DNA, 20 bp, Artificial Sequence
  RT-PCR primer set 2 forward for TSPAN2 transcript variant 2 (NM_001308315.1)
aacctacatgcccaaaggag

SEQ ID NO: 59 - DNA, 20 bp, Artificial Sequence
  RT-PCR primer set 2 reverse for TSPAN2 transcript variant 2 (NM_001308315.1)
atcatgccaaagatcgtcag

SEQ ID NO: 60 - DNA, 20 bp, Artificial Sequence
  RT-PCR primer set 3 forward for TSPAN2 transcript variant 2 (NM_001308315.1)
ctgacgatctttggcatgat

SEQ ID NO: 61 - DNA, 20 bp, Artificial Sequence
  RT-PCR primer set 3 reverse for TSPAN2 transcript variant 2 (NM_001308315.1)
acagctcctgtgacatttgg

SEQ ID NO: 62 - DNA, 20 bp, Artificial Sequence
  RT-PCR primer set 4 forward for TSPAN2 transcript variant 2 (NM_001308315.1)
aagaaagctccgaacaggtc

SEQ ID NO: 63 - DNA, 20 bp, Artificial Sequence
  RT-PCR primer set 4 reverse for TSPAN2 transcript variant 2 (NM_001308315.1)
cgatgcaattcttgtgtcct

SEQ ID NO: 64 - DNA, 20 bp, Artificial Sequence
  RT-PCR primer set 5 forward for TSPAN2 transcript variant 2 (NM_001308315.1)
aaaggcaatgggacactcat

SEQ ID NO: 65 - DNA, 20 bp, Artificial Sequence
  RT-PCR primer set 5 reverse for TSPAN2 transcript variant 2 (NM_001308315.1)
gacctgttcggagctttctt

SEQ ID NO: 66 - DNA, 20 bp, Artificial Sequence
  RT-PCR primer set 6 forward for TSPAN2 transcript variant 2 (NM_001308315.1)
tcaacatttcagtgctgtgg

SEQ ID NO: 67 - DNA, 19 bp, Artificial Sequence
  RT-PCR primer set 6 reverse for TSPAN2 transcript variant 2 (NM_001308315.1)
aagctcctttgggcatgta

SEQ ID NO: 68 - DNA, 20 bp, Artificial Sequence
  RT-PCR primer set 1 forward for TSPAN2 transcript variant 3 (NM_001308316.1)
aacctacatgcccaaaggag

SEQ ID NO: 69 - DNA, 20 bp, Artificial Sequence
  RT-PCR primer set 1 reverse for TSPAN2 transcript variant 3 (NM_001308316.1)
tctcgtgagtttcgtatcgc

SEQ ID NO: 70 - DNA, 20 bp, Artificial Sequence
  RT-PCR primer set 2 forward for TSPAN2 transcript variant 3 (NM_001308316.1)
tattcagcatggtcctctgc

SEQ ID NO: 71 - DNA, 20 bp, Artificial Sequence
  RT-PCR primer set 2 reverse for TSPAN2 transcript variant 3 (NM_001308316.1)
cagctcctgtgacatttggt

SEQ ID NO: 72 - DNA, 20 bp, Artificial Sequence
  RT-PCR primer set 3 forward for TSPAN2 transcript variant 3 (NM_001308316.1)
tcatgtgtgatctgcgtgtt

SEQ ID NO: 73 - DNA, 20 bp, Artificial Sequence
  RT-PCR primer set 3 reverse for TSPAN2 transcript variant 3 (NM_001308316.1)
ctgggagcgaaataggttgt

SEQ ID NO: 74 - DNA, 20 bp, Artificial Sequence
  RT-PCR primer set 4 forward for TSPAN2 transcript variant 3 (NM_001308316.1)
aagaaagctccgaacaggtc

SEQ ID NO: 75 - DNA, 20 bp, Artificial Sequence
  RT-PCR primer set 4 reverse for TSPAN2 transcript variant 3 (NM_001308316.1)
gcagaggaccatgctgaata

SEQ ID NO: 76 - DNA, 20 bp, Artificial Sequence
  RT-PCR primer set 5 forward for TSPAN2 transcript variant 3 (NM_001308316.1)
aaaggcaatgggacactcat

SEQ ID NO: 77 - DNA, 20 bp, Artificial Sequence
  RT-PCR primer set 5 reverse for TSPAN2 transcript variant 3 (NM_001308316.1)
gacctgttcggagctttctt

SEQ ID NO: 78 - DNA, 20 bp, Artificial Sequence
  RT-PCR primer set 6 forward for TSPAN2 transcript variant 3 (NM_001308316.1)
ggctgtatgttctggttgga

SEQ ID NO: 79 - DNA, 20 bp, Artificial Sequence
  RT-PCR primer set 6 reverse for TSPAN2 transcript variant 3 (NM_001308316.1)
aatatcaccaggaggcaggt

SEQ ID NO: 80 - DNA, 21 bp, Artificial Sequence
  RT-PCR primer set 7 forward for TSPAN2 transcript variant 3 (NM_001308316.1)
tggctcattactggtttaccc

SEQ ID NO: 81 - DNA, 20 bp, Artificial Sequence
  RT-PCR primer set 7 reverse for TSPAN2 transcript variant 3 (NM_001308316.1)
atcaggaattcccagcaaac

SEQ ID NO: 82 - DNA, 20 bp, Artificial Sequence
  RT-PCR primer set 8 forward for TSPAN2 transcript variant 3 (NM_001308316.1)
aagtacctgctgcttggctt

SEQ ID NO: 83 - DNA, 20 bp, Artificial Sequence
  RT-PCR primer set 8 reverse for TSPAN2 transcript variant 3 (NM_001308316.1)
tccgaaccgaaaccatagtc

SEQ ID NO: 84 - DNA, 20 bp, Artificial Sequence
  RT-PCR primer set 9 forward for TSPAN2 transcript variant 3 (NM_001308316.1)
tcaacatttcagtgctgtgg

SEQ ID NO: 85 - DNA, 20 bp, Artificial Sequence
  RT-PCR primer set 9 reverse for TSPAN2 transcript variant 3 (NM_001308316.1)
ctagaagctcctttgggcat

SEQ ID NO: 86 - DNA, 20 bp, Artificial Sequence
  RT-PCR primer set 10 forward for TSPAN2 transcript variant 3 (NM_001308316.1)
gcaatgtgtgcttggatcat

SEQ ID NO: 87 - DNA, 21 bp, Artificial Sequence
  RT-PCR primer set 10 reverse for TSPAN2 transcript variant 3 (NM_001308316.1)
tggtctgaacatgtcggatag

SEQ ID NO: 88 - DNA, 20 bp, Artificial Sequence
  RT-PCR primer set 11 forward for TSPAN2 transcript variant 3 (NM_001308316.1)
caaagaacaggcagtccatc

SEQ ID NO: 89 - DNA, 21 bp, Artificial Sequence
  RT-PCR primer set 11 reverse for TSPAN2 transcript variant 3 (NM_001308316.1)
gagttggaaagaacccgagta

SEQ ID NO: 90 - protein, 5 aa, Artificial Sequence
  anti-TSPAN2 antibody(IH00D) CDR-H1
NYWMN

SEQ ID NO: 91 - protein, 19 aa, Artificial Sequence
  anti-TSPAN2 antibody(IH00D) CDR-H2
EIRLKSNNYATHYAESVKG

SEQ ID NO: 92 - protein, 3 aa, Artificial Sequence
  anti-TSPAN2 antibody(IH00D) CDR-H3
GNY

SEQ ID NO: 93 - protein, 17 aa, Artificial Sequence
  anti-TSPAN2 antibody(IH00D) CDR-L1
KSSQSLLNSGNQKNYLA

SEQ ID NO: 94 - protein, 7 aa, Artificial Sequence
  anti-TSPAN2 antibody(IH00D) CDR-L2
GASTRES

SEQ ID NO: 95 - protein, 9 aa, Artificial Sequence
  anti-TSPAN2 antibody(IH00D) CDR-L3
QNDHSYPLT